(19) United States
(12) Patent Application Publication
(10) Pub. No.: US 2024/0109031 A1
(43) Pub. Date: Apr. 4, 2024

(54) SYSTEM FOR ACCELERATING DISSOLUTION OF MAFIC AND ULTRAMAFIC MATERIALS

(71) Applicant: PROJECT VESTA, PBC, San Francisco, CA (US)

(72) Inventors: Stephen J. ROMANIELLO, San Francisco, CA (US); Brian D. LEY, San Francisco, CA (US); Douglas O. EDWARDS, San Francisco, CA (US); Margaret G. ANDREWS, San Francisco, CA (US); Nathan G. WALWORTH, San Francisco, CA (US); Thomas ISHOEY, San Francisco, CA (US); Tom C. GREEN, San Francisco, CA (US); Francesc MONTSERRAT, San Francisco, CA (US); Martin VAN DEN BERGHE, San Francisco, CA (US); Kenneth NEALSON, San Francisco, CA (US); Devon Barnes COLE, San Francisco, CA (US); Kelly ERHART, San Francisco, CA (US)

(21) Appl. No.: 18/303,467

(22) Filed: Apr. 19, 2023

Related U.S. Application Data
(63) Continuation-in-part of application No. PCT/US22/78319, filed on Oct. 18, 2022.
(60) Provisional application No. 63/256,986, filed on Oct. 18, 2021.
(60) Provisional application No. 63/281,575, filed on Nov. 19, 2021.
(60) Provisional application No. 63/298,412, filed on Jan. 11, 2022.
(60) Provisional application No. 63/403,446, filed on Sep. 2, 2022.
(60) Provisional application No. 63/377,171, filed on Sep. 26, 2022.

(30) Foreign Application Priority Data
Feb. 17, 2022 (EP) 22157366.0

(51) Int. Cl. B01D 53/84; B01D 53/62; C02F 1/20; C02F 3/32; C02F 3/34; C12M 1/00

(52) U.S. Cl. CPC B01D 53/84; B01D 53/62; C02F 1/20; C02F 3/322; C02F 3/341; C02F 3/342; C12M 23/58; C12M 29/04; B01D 2251/402; B01D 2251/95; B01D 2257/504; B01D 2258/06; C02F 2101/10

(57) ABSTRACT

The present disclosure provides a system for processing ultramafic material. The system may comprise a reactor for accelerating weathering of said ultramafic material. The reactor may comprise one or more chambers comprising one or more microbes, biological medicators, or enzymatic accelerants to facilitate the weathering of the ultramafic material.

### CROSS REFERENCE TO RELATED APPLICATIONS

This application is a continuation-in-part of International Patent Application PCT/US22/78319, filed Oct. 18, 2022, which claims priority to U.S. Provisional Application No. 63/256,986, filed Oct. 18, 2021, U.S. Provisional Application No. 63/281,575, filed Nov. 19, 2021, U.S. Provisional Application No. 63/298,412, filed Jan. 11, 2022, U.S. Provisional Application No. 63/403,446, filed Sep. 2, 2022, U.S. Provisional Application No. 63/377,171, filed Sep. 26, 2022, and European Patent Application No. 22157366.0 filed Feb. 17, 2022; each of which is incorporated herein by reference in its entirety.

### BACKGROUND

Carbon dioxide is a powerful greenhouse gas, currently comprising 0.0415% (415 parts per million) of the Earth's atmosphere. Current anthropogenic emissions of carbon dioxide greatly exceed all available natural and manmade sinks, leading to persistent, long-term increases in the atmospheric concentration of carbon dioxide. The increase in atmospheric carbon dioxide concentration results in multiple deleterious effects on the natural environment, including rising global mean temperature, rising sea level, acidification of seawater, and changes in annual weather patterns, collectively known as climate change. There exists a need for systems, methods, compositions and processes to effect capture and sequestration of atmospheric carbon dioxide.

Certain naturally occurring geological minerals (e.g., olivine) as well as manmade industrial byproducts (e.g., slag) may chemically interact with carbon dioxide to effect the consumption of protons and the conversion of gaseous carbon dioxide into either aqueous dissolved bicarbonate and carbonate ions (HCO₃⁻ and CO₃²⁻) or solid-phase carbonate mineral species (CaCO₃₍ₛ₎ and MgCO₃₍ₛ₎), both of which act to remove carbon dioxide from the atmosphere (a process known as “Carbon Dioxide Sequestration”). Grinding these minerals to smaller particle sizes may increase the available surface area of such mineral particles, thereby enhancing the rate at which they are able to sequester carbon dioxide from the atmosphere (a process known as “Enhanced Weathering”.

### SUMMARY OF THE INVENTION

In one aspect, the present disclosure provides a system to address the Carbon Dioxide Sequestration needs. There exists a need to identify the appropriate properties (grainsize, color, density, hydraulic transport, etc.) of a suitably engineered material to use geological minerals (e.g., olivine or manmade industrial byproducts (e.g., slag) to remove carbon dioxide from the atmosphere (e.g., effect Carbon Dioxide Sequestration)). There also exists a need for methods and processes for the design, manufacture, utilization, or monitoring of such engineered material in real-world applications.

The availability and sustainable supply of valuable metals for the development of clean energy technologies, or “Technology Metals”, is paramount to reducing greenhouse gas emissions in the global economy. These metals may comprise those that are used to fabricate the critical components of numerous products and finished goods, including airplanes, automobiles, smart phones, and biomedical devices. For example, these can include rare-earth elements (REEs), platinum group metals (PGMs), lithium, copper, cobalt, silver, and gold. Large amounts of Technology Metals will be required for the sustainable development of clean technologies. Methods to concentrate and extract Technology Metals in seawater are critical to develop in order to scale this important and largely untapped source. There is also a need for the design and manufacturing of technologies to extract Technology Metals at increasing scales in real-world applications.

The system may comprise a bioreactor. The bioreactor may be configured to accelerate dissolution of mafic or ultramafic material (e.g., olivine). The bioreactor may be configured to facilitate the dissolution of mafic or ultramafic materials (e.g., olivine) in the presence of carbon dioxide to convert gaseous carbon dioxide into either aqueous dissolved bicarbonate and carbonate ions (HCO₃⁻ and CO₃²⁻) or solid-phase carbonate mineral species (CaCO₃₍ₛ₎ and MgCO₃₍ₛ₎).

In some embodiments, the bioreactor may comprise one or more mechanical components. The bioreactor may comprise a first chamber comprising the mafic or ultramafic material (e.g., olivine). In some embodiments, the mechanical components are coupled to the first chamber to induce a motion of the first chamber. The motion may be linear motion, rotational motion, or a combination thereof.

In some embodiments, the bioreactor may comprise a second chamber. The second chamber may comprise one or more microbes and/or microbial products (such as, but not limited to, live or dead microbial cells, and biomass), biological mediators (such as, but not limited to, extra-cellular ligands, chelators, nucleic or amino acids), and/or enzymatic accelerants (such as, but not limited to, extra- or intracellular metalloenzymes, oxidoreductases, transferases, hydrolases, ligases or isomerases). The one or more microbial products, biological mediators, and/or enzymatic accelerants may aid or facilitate the dissolution (e.g., weathering) of the mafic or ultramafic material (e.g., olivine). In some embodiments, the mafic or ultramafic material (e.g., olivine) may comprise pure olivine or a mixture of olivine and one or more other elements or materials, including sand. Such mafic or ultramafic material that includes a mixture comprising sand may form a carbon-removing sand. The olivine may be unprocessed (e.g., provided to the first chamber of the bioreactor in a form that is similar or identical to the naturally occurring form of olivine). In other cases, at least a portion of the olivine may be processed (e.g., provided to the first chamber of the bioreactor in a form that is altered or distinct from a naturally occurring form). Processed olivine may comprise olivine that has been grinded, or physically altered to change one or more physical characteristics of the olivine, including surface area, pore size, and density.

In some cases, the bioreactor may comprise a third chamber. The third chamber may comprise water. In some cases, the third chamber may be in fluid communication with a body of water. In some cases, the third chamber may further comprise at least a portion of water from the body of water. The body of water may optionally be a naturally occurring body of water such as an ocean, bay, lake, river, creek, stream, pond, marsh, wetland, swamp, or any other type of body of water. The body of water may be a marine body of water that may comprise salt water. The body of water may optionally comprise naturally occurring waves, current, flow, tides, or other activity.

Optionally, the bioreactor may comprise a fourth chamber. The fourth chamber may be in communication with the third chamber or any other chamber of the bioreactor. The fourth chamber may aid in extraction of Technology Metals from the third chamber or any other chamber of the bioreactor. Such extraction may occur by various chelating agents (e.g., various ligands), macrocycles (e.g., crown ethers and cyclams), cryptands, various separation materials (e.g., sorbents and membranes) and liquid extractants. In some embodiments, the Technology Metals may be separated by an extraction process and collected within the third or fourth chamber and/or be removed via one or more outlet and useful as described elsewhere herein.

In some embodiments, the first, second, third, and/or fourth chambers of the bioreactor are fluidically and operably coupled together through one or more channels or openings. In some cases, the contents of the chambers may combine and mix together. Any number of subset of chambers may be provided and/or communicating with one another. In some instances, such chambers may be provided in series and/or sequence. In some instances, chambers may be arranged in parallel.

To efficiently remove carbon dioxide from the atmosphere, at least one component of the carbon-removing sand maybe manufactured from an alkaline material. As used herein, such materials are referred to as “alkaline materials” and may refer to one or more entries of Table 1:

Naturally Occurring Rocks: Peridotite, Dunite, Harzburgite, Lherzolite,
Basalt, Basaltic Glass, Gabbro, Komatiite, Serpentinite, Kimberlite
Naturally Occurring Minerals: Olivine, Forsterite, Fayalite, Enstatite,
Pyroxene, Serpentine, Brucite, Wollastonite
Industrial Byproducts: Blast-furnace Slag, Steel-furnace Slag, Basic-
oxygen-furnace Slag, Electric-arc-furnace Slag, Ladle Slag.

Not limiting examples of alkaline materials may comprise olivine, dunite, basalt, serpentinite, serpentine, brucite, wollastonite, or industrial-produced mineral-equivalents such as slag or mine tailings. These minerals interact with water and carbon dioxide and/or carbonic acid to produce bicarbonate or carbonate ion or solid carbonate precipitate as a product, thereby decreasing the acidity of the surrounding fluid and converting the harmful carbon dioxide or carbonic acid into environmentally beneficial bicarbonate or carbonate form. This reaction typically occurs on decadal or centennial timescales, thereby rendering it sufficient for climate mitigation and effectively inert on the instantaneous environment. In some instances, carbonate precipitates may function as a form of carbon storage. An example describing the interaction of forsterite olivine (Mg₂SiO₄) with carbon dioxide (CO₂) dissolved in seawater is provided below, although other minerals and rocks described in this disclosure may result in equivalent reactions converting dissolved carbon dioxide and water (carbonic acid) to bicarbonate ion:

Mg₂SiO₄+4CO₂+4H₂O→2Mg²⁺+4HCO₃⁻+H₄SiO₄ 1.

Through the conversion of carbon dioxide to dissolved bicarbonate and carbonate ions, this reaction acts to reduce the partial pressure of carbon dioxide in seawater. In coastal construction projects, this seawater is in close contact with the surface ocean and atmosphere, allowing net transfer of carbon dioxide across the air-sea interface, thereby effecting the net sequestration of atmospheric carbon dioxide as bicarbonate and carbonate ion in seawater.

In some embodiments, the one or more chambers of the bioreactor are amended with certain chemicals that can serve to enhance carbon capture (such as and not limited to, fertilizers or carbon dioxide from flue gasses, various plastics). In one example, increased carbon dioxide concentrations in the medium may also increase growth rate of microbes as described herein.

Another aspect of the invention relates to processes and methods for quantifying rate and extent to which carbon-removing sand sequesters carbon dioxide. These methods may include determination of the concentration, flux, or isotopic composition of chemical species resulting from the dissolution of carbon-removing sand. These methods may also include determination of the impact of carbon-removing sand upon the ambient concentration, flux, or isotopic composition of gaseous or aqueous carbon dioxide species and alkalinity found in the region surrounding carbon-removing sand.

In another aspect, these methods may optionally include the introduction of a chemical or isotopic tracer which serves to facilitate the determination of the rate or extent at which carbon removing sand undergoes chemical dissolution or transformation.

In another aspect, these methods may be conducted at a single point in time or as part of a time series.

In one embodiment, these determinations are made in the pore fluid in contact with the carbon-removing sand, as well as the overlying water. In other embodiments, these determinations may be made via the installation of a chamber installed upon the sediment surface which acts to integrate the accumulation of reaction products and/or the depletion of chemical reactants across the sediment-water interface.

In still other embodiments, these methods may include determining the flux of aqueous or gaseous carbon dioxide in the overlying air or water by means of eddy covariance techniques.

In still further embodiments, these methods may include determination of the rate of dissolution or chemical transformation of the carbon-removing sand material via quantification of the abundance of the initial and subsequent mineral and organic phases present in sediments.

While all the above methods may be conducted in situ, in still other embodiments any or all of the above methods may be conducted ex-situ via the construction of a reactor apparatus which serves to emulate the behavior of carbon-removing processes in the environment. Such reactors may be conducted at a range of sizes and scales including but not limited to laboratory “bench-scale” reactors, batch-scale reactors, larger outdoor mesocosm scale reactors or other reactors designed to replicate desired real-world conditions in certain embodiments. Such reactors may optionally be constructed in such a manner as to make them portable facilitating transportation between sites.

In some embodiments of the invention, determination of the rate at which carbon-removing processes reactions may be facilitated, predicted, or summarized via the construction of a mathematical computer model. Such a model may accept certain environmental, biological parameters and/or physical properties of the carbon-removing processes and/or the results of the aforementioned chemical, biological or physical determinations to output either the dissolution rate of carbon-removing processes and/or the physical and chemical impact of carbon-removing sand on the surrounding environment.

In some embodiments of the invention, Technology Metals including but not limited to nickel and cobalt may be extracted from the bioreactor chambers or from additional chambers downstream of the third chamber. Extraction methods may include but are not limited to various chelating agents (e.g., various ligands), macrocycles (e.g., crown ethers and cyclams), cryptands, various separation materials (e.g., sorbents and membranes) and liquid extractants.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates the long-term carbonate-silicate cycle, in accordance with some embodiments.

FIG. 2 schematically illustrates the chemical process by which olivine can be used to capture and sequester carbon dioxide, in accordance with some embodiments.

FIG. 3 schematically illustrates the chemical reactions that enable carbon dioxide capture and sequestration using olivine, in accordance with some embodiments.

FIG. 4 schematically illustrates an example of how the oceans can be used for natural carbon storage, in accordance with some embodiments.

FIG. 5 schematically illustrates an example of a bioreactor, in accordance with some embodiments.

FIG. 6 schematically illustrates a computer system that is programmed or otherwise configured to implement methods provided herein.

FIG. 7 schematically illustrates an exemplary reactor configured to intake seawater that may be mixed with olivine and microbial biomass to generate alkalinity.

FIG. 8 schematically illustrates a chemical reaction by which Coastal Carbon Capture occurs when olivine is introduced to a target site, in accordance with some embodiments.

FIG. 9 schematically illustrates a chemical reaction by which coastal carbon capture occurs when olivine is introduced to a target site and highlights that bicarbonate ions contribute to ocean alkalinity, in accordance with some embodiments.

FIG. 10 schematically illustrates a plot of concentration of an alkaline material in a flux chamber as a function of time, in accordance with some embodiments.

FIG. 11 schematically illustrates a plot of concentration of an alkaline material in a flux chamber as a function of time as compared to a control site, in accordance with some embodiments.

FIG. 12 schematically illustrates an overview of the porewater method, in accordance with some embodiments.

FIG. 13 schematically illustrates a shrinking core model, in accordance with some embodiments.

FIG. 14 schematically illustrates dissolution rates for olivine having various grain sizes, in accordance with some embodiments.

FIG. 15 schematically illustrates an exemplary modeling approach for particle distribution, in accordance with some embodiments.

FIGS. 16-17 schematically illustrate various examples of sensors that can be used for measuring, recording, and verifying carbon capture and/or olivine dissolution, in accordance with some embodiments.

FIG. 18 schematically illustrates various plots showing exemplary olivine dissolution kinetics for olivine particles having different grain sizes, in accordance with some embodiments.

FIG. 19 schematically illustrates the effects of secondary carbonate precipitation on carbon dioxide capture efficiency, in accordance with some embodiments.

FIG. 20 schematically illustrates the effects of secondary clay formation on carbon dioxide capture efficiency, in accordance with some embodiments.

FIG. 21 schematically illustrates Earth's natural carbon dioxide removal process as compared to an accelerated weathering process, in accordance with some embodiments.

FIG. 22 schematically illustrates a process overview of a bioreactor, in accordance with some embodiments.

FIG. 23 schematically illustrates that abiotic olivine dissolution rates in the first 50 hours can be over an order of magnitude greater than the long-term, sustained dissolution rates, which become asymptotic.

FIG. 24 shows biofilm growth on olivine grains in accordance with some embodiments of the present disclosure.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “real time” or “real-time,” as used interchangeably herein, generally refers to an event (e.g., an operation, a process, a method, a technique, a computation, a calculation, an analysis, a visualization, an optimization, etc.) that is performed using recently obtained (e.g., collected or received) data. In some cases, a real time event may be performed almost immediately or within a short enough time span, such as within at least 0.0001 millisecond (ms), 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1 ms, 0.05 ms, 0.01 ms, 0.005 ms, 0.001 ms, 0.0005 ms, 0.0001 ms, or less.

### Overview

To avoid the worst effects of climate change, we must rapidly remove billions of tons of carbon dioxide from the atmosphere. There is an urgent need to identify carbon removal methods which are permanent, scalable, and economical.

The earth's long-term carbonate-silicate cycle is how our planet has naturally captured carbon dioxide from the atmosphere. Over millennia, rain falling on exposed igneous rock causes such rocks to slowly dissolve in a process known as “weathering.” Carbonic acid dissolved in the rainwater reacts with silicate from such rocks, generating alkalinity and shifting equilibrium from carbonic acid to bicarbonate. This water eventually flows to the oceans, which ultimately causes the ocean to absorb carbon dioxide from the atmosphere as bicarbonate dissolved in ocean water. Bicarbonate has a long ocean residence time, significantly longer than human timescales, with any subsequent biotic or abiotic precipitation of carbonate minerals resulting from increased bicarbonate causing the formation of carbonate rock.

“Carbon capture bioreactors” refers to the class of negative biological and geochemical carbon dioxide emissions technologies (NETs) which seek to remove atmospheric carbon dioxide and store it on human timescales through the acceleration of the natural weathering processes. Carbon Dioxide Sequestration may occur through the generation of alkalinity (e.g., bicarbonate) or the formation of carbonates.

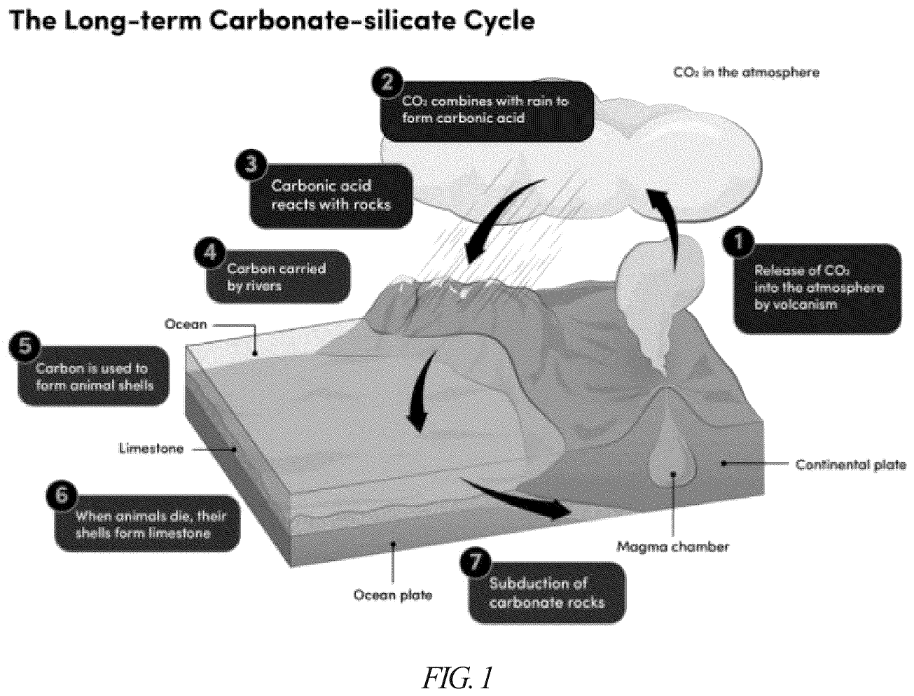

FIG. 1 illustrates the long-term carbonate-silicate cycle. Natural carbon dioxide removal through rock weathering may be achieved through the following steps:
1. Rain falls on igneous rocks, slowly dissolving them.
2. Carbonic acid dissolved in the rainwater reacts with silicate from such rocks, generating alkalinity and shifting the equilibrium from carbonic acid to bicarbonate.
3. This bicarbonate flows to the ocean.
4. The bicarbonate combines with calcium and magnesium ions to form carbonate.
5. Carbonate is deposited on the seafloor, thereby capturing atmospheric carbon dioxide in rock.

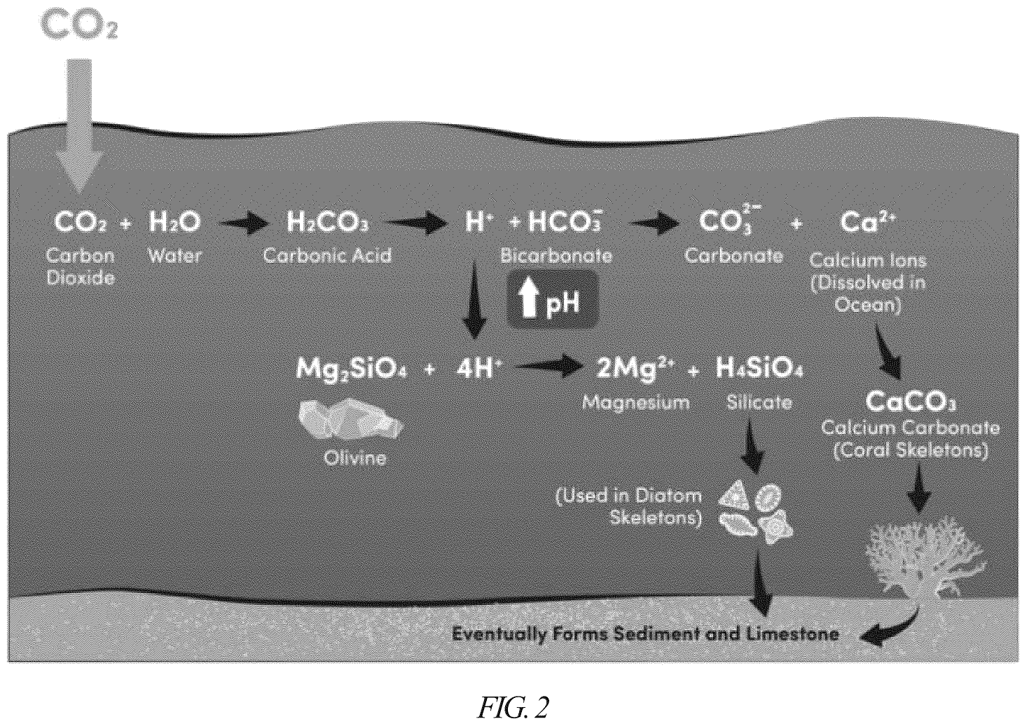

FIG. 2 illustrates the chemical processes which occurs as part of the long-term carbonate-silicate cycle involving mafic or ultramafic materials resulting in carbon dioxide capture and sequestration, i.e. the conversion of dissolved carbon dioxide and water to bicarbonate via olivine, which thereby allows uptake of atmospheric carbon dioxide into the surface ocean and seawater pH increase.

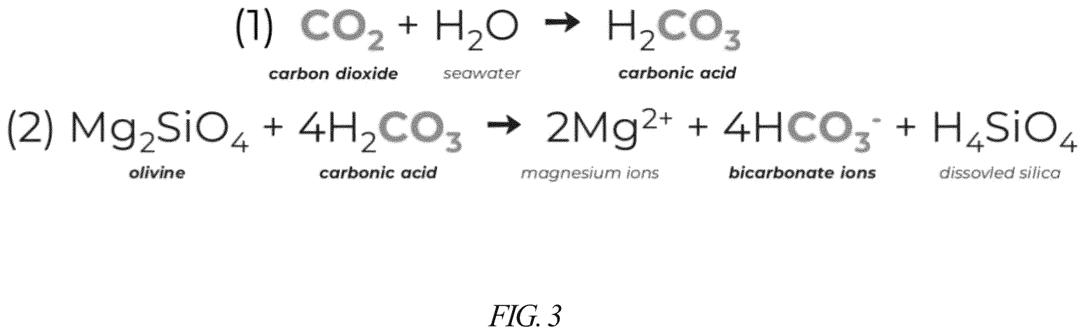

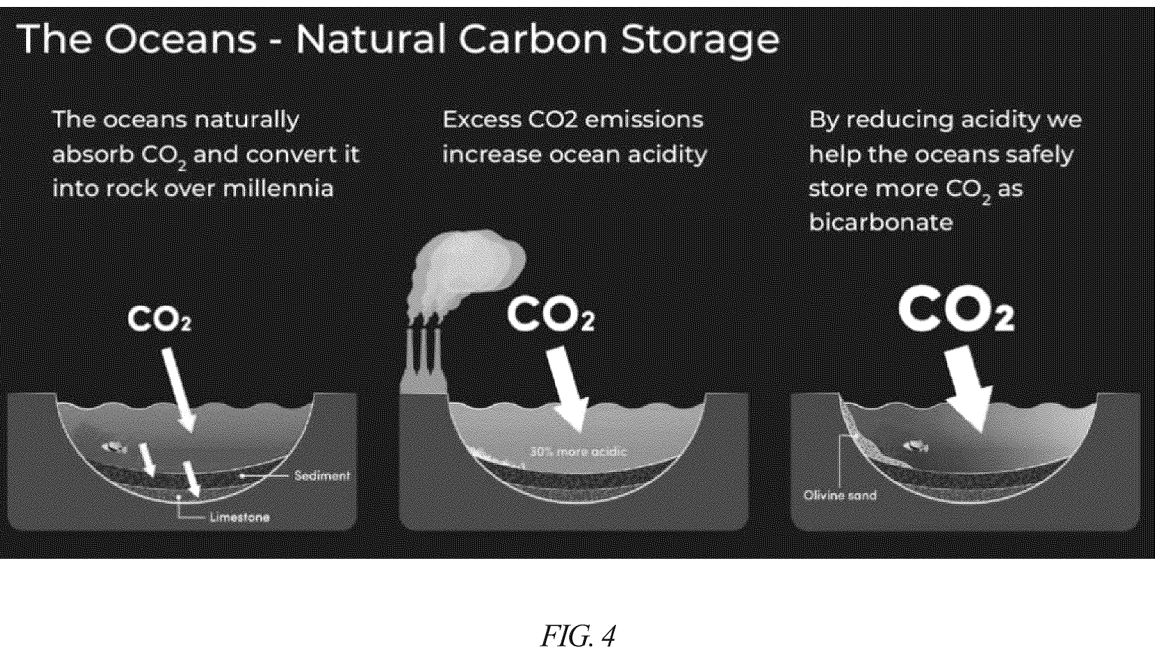

FIG. 3 illustrates a chemical process that enables carbon dioxide capture and sequestration using olivine. As the olivine dissolves, its products are magnesium ions (the second most abundant ion in the ocean behind sodium), silicate (used by diatoms to build their skeletons) and dissolved carbon. Referring to FIG. 2 and FIG. 3, when olivine dissolves in water, it drives the illustrated reaction, thus increasing carbon dioxide uptake, raising pH, and generating alkalinity.

As a result, this process has the potential co-benefit of counteracting ocean acidification. Ocean acidification is the process by which increasing atmospheric carbon dioxide dissolves in seawater, which reduces pH (increasing acidity) (upper reaction in diagram below). This reduces the ability of calcifying organisms like corals to grow and produce exoskeletons, or shells. As shown in FIG. 2 and FIG. 3, dissolving alkaline material (e.g., olivine) in water sequesters hydrogen ions into dissolved silicate (H₄SiO₄), a molecule that can be used by diatoms-important photosynthesizing algae that fix carbon dioxide and form the base of the marine food web.

Unfortunately, natural chemical weathering happens too slowly to correct for human carbon dioxide emissions on human-relevant timescales. This natural process is also already accounted for in Earth's present-day carbon budget. The systems and methods disclosed herein can be deployed and implemented to accelerate this natural process to remove at least one additional gigaton of atmospheric carbon dioxide per year on a global scale.

### Bioreactor

In an aspect, the present disclosure provides a reactor configured to maximize carbon capture via the accelerated dissolution of a mafic or ultramafic material (e.g., olivine) for weathering applications. In some cases, the reactor may comprise or be a bioreactor.

In another aspect, the present disclosure provides systems and methods to accelerate olivine dissolution reactions in a bioreactor. The bioreactor may comprise one or more internal chambers (e.g., a first chamber, a second chamber, and/or a third chamber). One or more of such chambers (e.g., the first chamber) may house or store olivine for an amount of time. The amount of time may be greater than or equal to about 1 minute, 30 minutes, 1 hour, 5 hours, 12 hours, 1 day, 5 days, 10 days, 50 days, 100 days, 1 month, 3 months, 6 months, 1 year, 2 years, 5 years, 10 years or longer. The amount of time may be less than or equal to about 10 years, 5 years, 2 years, 1 year, 6 months, 3 months, 1 month, 100 days, 50 days, 10 days, 5 days, 1 day, 12 hours, 5 hours, 1 hour, 30 minutes, 1 minute or less. The amount of time may be between any two of the amounts of time described above, for example between about 1 day and about 3 months.

The bioreactor may comprise one or more openings or inlets for receiving olivine. The one or more opening or inlets may be operably coupled to a first chamber of the bioreactor. The one or more openings or inlets may be further coupled to a source of olivine. The olivine may comprise pure olivine or a mixture of olivine and sand or other elements or materials. The olivine may be unprocessed. Alternatively, the olivine may be processed or pre-processed (e.g., by grinding). In some cases, grinding these minerals to smaller particle sizes may increase the available surface area of such mineral particles, thereby enhancing the rate at which they are able to sequester carbon dioxide from the atmosphere (a process known as “Enhanced Weathering”).

In some cases, the bioreactor may comprise one or more mechanical components for mechanical weathering of the olivine. The one or more mechanical components may be operably coupled to one or more chambers of the bioreactors. For instance, the one or more mechanical components may be operably coupled to a first chamber, second chamber, third chamber, and/or fourth chamber of a bioreactor. The one or more mechanical components may be operably coupled to a power system, drive train, or mechanical motor. The mechanical components may be powered by the power system, drive train, or mechanical motor to create a motion of or in one or more of the chambers of the reactor. For example, a rotary mechanical component may be coupled to a geared drive train. The rotary mechanical component may rotate within the first chamber of the bioreactor, which may comprise the olivine materials. The rotary mechanical component may introduce mechanical energy into the olivine materials, which may accelerate the dissolution of the olivine material within the first chamber. The concept of introducing mechanical energy into olivine material within a chamber of the bioreactor may be referred to as “mechanical weathering.”

The one or more mechanical components may permit mixing within a chamber and/or between contents of different chambers. For example, one or more mechanical components may facilitate mixing between contents of first and second chamber, and/or between a second and third chamber and/or between a first and third chamber. One or more mechanical components may comprise pumps, or systems configured to provide fluid manipulation to transfer mechanical energy to the contents of the chamber. In some instances, systems configured to provide fluid manipulation are configured for fluid filtration, sterilization, cavitation, flow control, particulate recovery, flocculation, centrifugation, phase change reactions, pressurization or de-pressurization, fluid mixing, fluid separation, heat transfer, pressure release, ventilation, or management of sub-, trans-, super- and hyper-sonic fluid flows.

Optionally, one or more mechanical components are operably coupled to a body of water within a zone comprising wave or current energy, such that said one or more mechanical components transfer mechanical energy from said body of water to said contents of one or more chambers. In some instances, the bioreactor may be at or near a naturally occurring body of water that may effect naturally occurring wave or current enter. Thus, environmental activity may be used to facilitate transfer of mechanical energy within one or more chambers of the bioreactor. This may allow for naturally occurring energy to be used to facilitate weathering, mixing, heat exchanges, and/or other activities within the bioreactor.

The mechanical weather may cause grain-on-grain collisions between grains of olivine material that may rapidly break down the olivine. Olivine grains may be reduced in size due to mechanical activation.

In some cases, the bioreactor may be in fluid communication with water. In some cases, the bioreactor may comprise an internal chamber for housing the water and/or the olivine. The one or more mechanical components may be coupled to a chamber of the bioreactor comprising water, such that operation of the mechanical components may cause a motion of the water within the chamber. The motion of the water, when combined with olivine, may cause surface abrasions of the olivine and accelerate the rate of dissolution of the olivine. In some cases, the surface of the olivine grains may be mechanically activated to enable and/or enhance carbon dioxide uptake and ocean de-acidification.

In some cases, the mechanical components may comprise one or more gears or actuators for inducing a motion that cause mechanical weathering of the olivine. The motion may be a linear motion, or a rotational motion, or any combination thereof.

In some cases, the bioreactor may comprise a chamber for housing one or more microbial products or other biological mediators and/or enzymatic accelerants that can help to further break down the olivine. The chamber housing the microbial products may be in fluid communication with (i) the chamber housing the olivine, (ii) the chamber that is in fluid communication with the ocean water, or both (i) and (ii).

In some cases, the bioreactor may comprise a chamber to extract Technology Metals. Extraction methods may include but are not limited to various chelating agents (e.g., various ligands), macrocycles (e.g., crown ethers and cyclams), cryptands, various separation materials (e.g., sorbents and membranes) and liquid extractants.

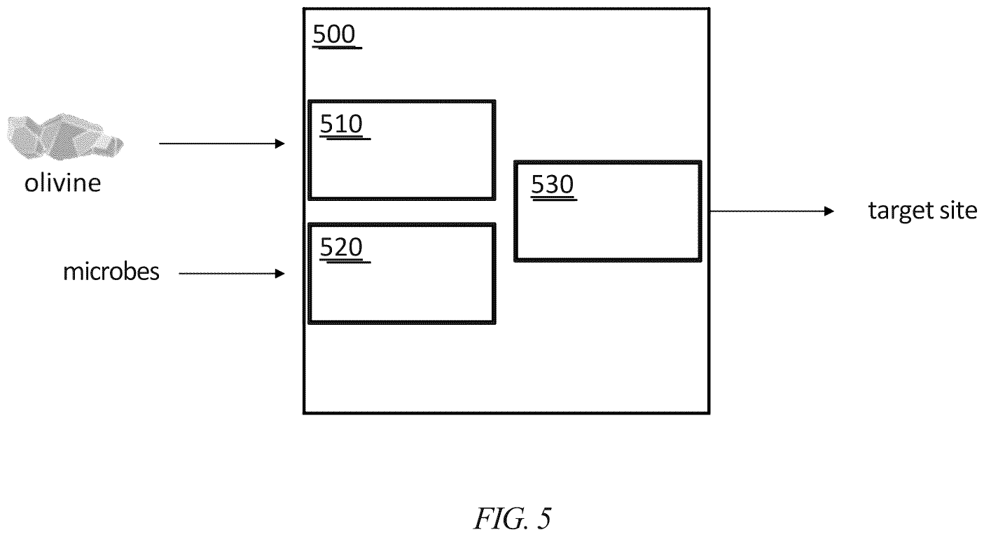

FIG. 5 schematically illustrates an example of a bioreactor 500. The bioreactor 500 may be configured to receive olivine, which may or may not be pre-processed (e.g., by grinding or crushing). The olivine may be stored within a first chamber 510 of the bioreactor 500 for further processing. In some cases, the bioreactor 500 may comprise a second chamber 520 configured to house one or more microbial products or other biological mediators and/or enzymatic accelerants that can help to further break down the olivine. The chamber may comprise one or more microbes and/or microbial products (such as, but not limited to, live or dead microbial cells, and biomass), biological mediators (such as, but not limited to, extra-cellular ligands, chelators, nucleic or amino acids), and/or enzymatic accelerants (such as, but not limited to, extra- or intracellular metalloenzymes, oxidoreductases, transferases, hydrolases, ligases or isomerases).

Examples of microbes and/or microbial products may include, but are not limited to bacteria, archaea, protozoa, fungi, algae, lichens, slime molds, viruses, and/or prions. One or more microbes and/or microbial products may comprise live or dead microbial cells or biomass, which may comprise heterotrophic, chemolithoauotrophic or photoautotrophic prokaryotes or eukaryotes. One or more microbes or microbial products may comprise bacteria that are capable of growth in aqueous mediums. Examples of such bacteria may include Alteromonas sp., Shewanella sp., Marinobacter sp., Pseudomonas sp., Synechococcus sp., and Synechocystis sp. The microbes or microbial products may comprise eukaryotic algae capable of uptake of bacterially produced siderophores. In some instances, eukaryotic algae may comprise Phaeodactylum sp., Fragilaria sp., or Thalassiosira diatom species. Optionally, the microbes and/or microbial products may comprise marine microbes capable of growing in saltwater.

In some cases, the bioreactor 500 may comprise a third chamber 530. The third chamber 530 may comprise a reaction chamber in fluid communication with the first chamber 510 and/or the second chamber 520. In some instances, mixing may occur between the contents of the first and/or second chamber, with the third chamber. The third chamber 530 may be used to break down the olivine using the microbial products or other biological mediators and/or enzymatic accelerants. Once broken down, the processed olivine may be provided to a target region external to the bioreactor 500 to facilitate carbon dioxide capture and sequestration.

### Microbes

[2] In another aspect, the present disclosure provides a method for enhanced dissolution of olivine through (1) microbial products or other biological mediators and (2) enzymatic accelerants. The method may comprise enhancing dissolution of olivine in an olivine reactor or sand pit resulting from coastal nourishment projects or dredging through microbial products or other biological mediators, and enzymatic accelerants.

In another aspect, the present disclosure provides systems and methods for olivine dissolution by microorganisms, biological organisms, and/or biological mechanisms. The dissolution of olivine in water may consume protons and increase alkalinity, thereby removing dissolved aqueous carbon dioxide. This aqueous carbon dioxide removal may induce additional carbon dioxide to be taken up from the atmosphere and subsequently converted to bicarbonate, carbonate, and/or solid-phase carbonate minerals, thereby sequestering carbon for long time periods [1]. Scaling and accelerating this process has the potential to mitigate climate change and ocean acidification.

In some cases, the reactors disclosed herein may be designed to grow particular microbial strains in an aqueous medium in order to induce metabolic states that can accelerate olivine dissolution. In circum-neutral pH naturally oxygenated waters, ferric iron (Fe²⁺) contained within olivine may be rapidly oxidized to ferrous iron (Fe³⁺) by oxygen even in the absence of microbial activity [2]. This oxidized iron can form a surface layer of Fe-oxyhydroxides on the mineral surface of olivine which can shield portions of its total surface area from contact with solution [3]. These Fe-oxyhydroxide surface coatings can, in some cases, impede olivine dissolution [4]. In some cases, a layer ferric oxy-hydroxide forms around an olivine particle as it weathers. In some cases, the layer of ferric oxy-hydroxide on an olivine particle has a thickness of less than 50 nanometers, less than 25 nanometers, less than 20 nanometers, less than 15 nanometers, less than 10 nanometers, less than 5 nanometers, less than 3 nanometers, less than 2 nanometers, less than 1 nanometer or smaller. In some cases, catalysts may be used to remove at least a portion of a ferric oxy-hydroxide layer on an olivine particle. In some cases, other methods to reduce or remove a ferric oxy-hydroxide layer may improve the feasibility and scale of carbon dioxide removal through olivine weathering.

For the vast majority of organisms, Fe is a required nutrient and a key component of many cellular proteins. However, most Fe in naturally oxygenated waters at circum-neutral pH is insoluble and typically complexed to particles. Certain microorganisms can produce organic compounds known as siderophores to mobilize and acquire insoluble iron on mineral surfaces [2], which can concurrently increase olivine mineral dissolution rates [3]. However, the production of siderophores may be regulated by Fe availability to the cells. As more Fe is acquired by the cells (i.e. the less iron-limited they are), siderophore production may decrease, which may slow dissolution rates. Hence, in order to sustain and scale siderophore production over extended time periods in large reactors, numerous interacting factors may be optimized or improved:
1. Advantageous biological strains may be identified through a series of experiments, and in some cases, custom strains may be generated either through directed evolution or genetic editing. These strains are ones capable of accelerating dissolution through the biological production of organic ligands such as, but not limited to, siderophores. These microbes include certain species of bacteria like Alteromonas sp. and Shewanella sp. that can grow in aqueous mediums. These microbes can include eukaryotic algae that can uptake bacterially produced siderophores such as Phaeodactylum sp. and Thalassiosira sp. diatom species.
2. The optimal or beneficial ratios of microbial to siderophore concentrations given certain growth conditions. In some instances, beneficial ratios may include about 100:1, 50:1, 20:1, 10:1, 5:1, 3:1, 2:1, 1:1, 1:2, 1:3, 1:5, 1:10, 1:20, 1:50, or 1:100. In some cases, the beneficial ratios may be between two of the values described above, for example the beneficial ratio of microbial to siderophore concentrations may be between about 10:1 and about 1:10.
3. Olivine dissolution optimization as a function of particle size/surface area and flow rate from biomass generation.
4. Optimized long-term growth conditions for sustained siderophore production.
5. Optimized reactor design series including appropriate materials to avoid silicate and trace metal contamination.
6. Development of mathematical models to optimize siderophore production yield, biomass generation, and olivine dissolution.
7. Development of appropriate parameters to track to feed into the model.
8. Development of any treatment of seawater effluent prior to returning to the ocean.
9. Development of a model that calculates carbon dioxide sequestration from the addition of alkalinized seawater to the local ocean region.

Siderophores are organic ligands with extremely high binding affinity for Fe(III) iron ions and intermediate affinities for Fe(ii), Mn(III) ions, and many other transition metals. In some cases, siderophores described and used in systems and methods described herein may be produced by bacteria and other fungi. The bacteria or fungi may produce siderophores as a component of a high-affinity iron uptake system. Bacteria and fungi that exist in aerobic marine environments may have a higher affinity for producing such siderophores due to the concentration of soluble iron. In some environments, iron has a calculated solubility of about 10⁻¹⁶M.

One of example of a siderophore as described herein is putrebactin, produced by Shewanella oneidensis. Some embodiments of compositions described herein may comprise Shewanella oneidensis and/or putrebactin. Shewanella oneidensis may obtain nutrients for growth from olivine particles. Production of putrebactin significantly increases the rate of olivine dissolution, and corresponding obtainment of nutrients of the olivine particles for growth of bacteria (e.g. Shewanella oneidensis). Other examples of siderophores can include but are not limited to petrobactin produced by Alteromonas, enterobactin, the Desferrioxamine family, and Rhizoferrin.

Olivine dissolution rates may increase by over an order of magnitude or more via siderophore synthesis. Several key biological and engineering improvements may increase the viability of carbon dioxide removal (CDR) by microbially mediated mineral dissolution, implemented at scale.

### The Biological Underpinnings of Mineral Dissolution

The precise mechanisms involved in the microbial acquisition of particulate-bound nutrients may include Siderophore activity. Siderophores can play a crucial role in transforming mineral-phase iron (esp. oxidized iron) into a bioavailable form, which can remove a thin (nanometer-thick) layer of oxidized iron deposited along the surface of olivine grains. This process enables microbial exponential growth for siderophore-producing bacteria, whereas bacteria incapable of producing siderophores may be unable to grow in the same conditions. In some cases, this accelerated dissolution is most pronounced during microbial exponential growth, which would be the period of maximal demand for new iron entering the biological pool. Therefore, in some embodiments, the methods of dissolving olivine may comprise providing at least one bacteria strain (e.g., Shewanella oneidensis) to produce at least one siderophore (e.g., putrebactin) to increase a rate of dissolution of a composition comprising olivine.

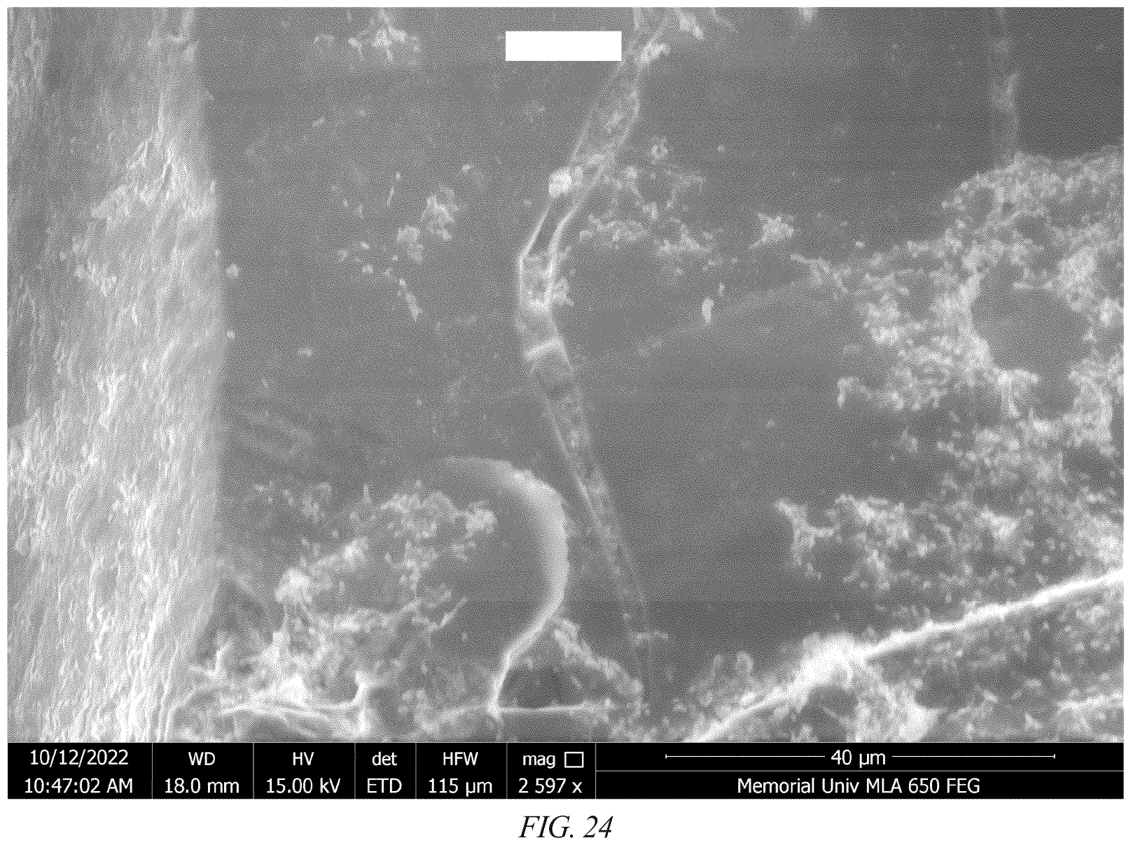

In some cases, siderophore activity can be augmented by other, complementary microbial processes related to the formation of biofilms along mineral surfaces. Bacteria can form thick, redox-active biofilms along mineral surfaces, shown in FIG. 24, that can support abundant and dense growth. While in some cases biofilms can inhibit mineral dissolution, the success of these biofilms in sustaining growth in environments where dissolved nutrients are limiting suggests active mechanisms of nutrient acquisition directly from mineral phases. Biofilms can host a wide range of metabolic activities. If this activity can be channeled towards mineral-phase nutrient acquisition, the sheer density and intimate proximity of biofilms to mineral surfaces can have a profound effect on mineral dissolution rates. In addition to siderophore activity, respiration can significantly lower the pH along the mineral surface, further enhancing mineral dissolution rates. Bacteria can also produce significant amounts of organic acids within the confines of biofilms (e.g., lactate, pyruvate, and citrate) which can have significant effects on localized pH, and are often excellent cation sorbents that can further help promote mineral dissolution. Other ligands that may accelerate olivine dissolution can include but are not limited to Ferritin and oxalate.

In some cases, a genetic and physiological understanding of mineral dissolution mechanisms would critically inform the utilization and application of microbially enhanced dissolution reactions. Identification of genetic synthesis pathways and their regulators may inform engineering efforts to improve dissolution. In some embodiments, one or more methods described herein may comprise determining or identifying a genetic synthesis pathway. The method may further comprise determining or identifying one or more regulators of the genetic synthesis pathway. The method may further comprise using the determination or identification of the pathways and/or one or more regulators to engineer the pathway to be over expressed, under expressed, or to express an alternative gene. Particularly, a mechanistic understanding may drive selection of promising organisms for large scale dissolution systems, the application of genetic engineering to maximize dissolution, and the design of culture environments and reactor systems. A method for selecting organisms for large scale dissolution systems may comprise understanding one or more mechanisms of ultra-mafic particle (e.g., olivine) dissolution via one or more biological pathways. In another embodiment, a method for genetic engineering of one or more biological species (e.g., bacteria) may comprise understanding one or more mechanisms of ultra-mafic particle (e.g., olivine) dissolution via one or more biological pathways. In another embodiment, a method for designing one or more culture encironements and/or reactor systems may comprise understanding one or more mechanisms of ultra-mafic particle (e.g., olivine) dissolution via one or more biological pathways. In some cases, understanding one or more mechanisms of ultra-mafic particle (e.g., olivine) dissolution via one or more biological pathways may comprises basing a design of an environment or reactor, genetically modifying one or more biological species, or selecting organisms for large scale dissolution systems based on at least some information determined from an analysis of the one or more mechanisms for ultra-mafic particle (e.g., olivine) dissolution.

### The Upper Limit of Microbially Mediated Dissolution and Biosynthetic Production

Sustaining microbially-accelerated mineral dissolution may include a continuous maintenance of an environment that favors both a specific metabolic activity from a microbiome, as well as the kinetics and thermodynamics of mineral dissolution and carbon drawdown. In some embodiments, the reactor conditions may maintain an environment in a steady geochemical state to promote a specific reaction. In some embodiments, these reactor conditions may reflect sea surface conditions at pressures of approximately 1-2 bar and temperatures ranging from 0-40 degrees Celcius. In some embodiments, olivine and biological component (e.g., microorganisms, enzymes, etc.) concentrations may be maintained at ratios of 0.00001-0.005 kg to 10³-10¹⁰ milliliter of fluid. In some embodiments, specific environmental variables or additives may be optimized or added to the bioreactor including but not limited to temperature, light, nutrients, flocculants, or surfactants. In some embodiments, biological strains may be genetically engineering to better dissolve the mafic materials. In some cases, biomes contained within a reactor of the present disclosure may contain a plurality of natural or genetically engineered microbes.

Microorganisms may also add separate economic value beyond generation of alkalinity from mineral dissolution. This economic value can include biosynthesis of lipids, vitamins, and other biomolecules that have societal and economic value in industries such as pharmaceuticals, or agri- and aquaculture. Alternatively, certain substrates introduced into the reactors can promote the growth of a specific strain or metabolism, such as certain degradable plastics (e.g., PHA's and PHB's) as a carbon source for biofilm-forming heterotrophs. In some cases, specific substrates may be selected for specific, desired microbial activity, and can offer a sustainable means to manage plastic wastes while replacing the costs of adding a carbon source to the reactor. Lastly, some processes described herein may involve the dissolution of large quantities (i.e., gigatonnes) of various mineral substrates of different grain sizes that may release significant amounts of trace metals associated with ultramafic materials, most notably nickel, cobalt, and chromium. These metals are particularly valuable, as they play a critical role in the transition away from fossil fuels and towards an electricity-based economy.

In one aspect, microorganisms can be genetically engineered for large-scale mineral dissolution processes, specifically for production of siderophores, formation of biofilms, production of valuable biomolecules, and growth on renewable substrates. Identification of the genetic networks responsible for these phenotypes represents the enabling technology to enhance or tune strain performance.

Siderophore synthesis pathways have been identified in many organisms through both experiments and bioinformatics. Several pathways have been expressed and further engineered in the model organism Escherichia coli, with the goal of producing siderophores as therapeutic antibiotics. To our knowledge, there have been no, or limited, efforts to enhance the production of endogenous siderophores, to date. Siderophores are typically produced in bacteria by non-ribosomal peptide synthesis and secretion, which are both resource and energy intensive processes. Enhanced production may require constitutive expression of siderophore biosynthesis systems both endogenously and heterologously, metabolic engineering for increased availability of siderophore precursors, or alterations of the bacterial secretion complex. Transcriptomic studies may be performed on siderophore-producing organisms under iron-limiting conditions. However, the biological bottleneck for production of siderophores is often complex. For example, S. aureus can produce both staphyloferrin A (SA) or B (SB), both of which are citrate based siderophores; downregulation of the TCA during Fe limitation limits the production of SA due to decreased citrate synthase activity. An alternate citrate synthase upregulated by Fe limitation supports synthesis of SB however. A method of transcriptionally and metabolically maintaining enhanced siderophore synthesis may comprise one or more elements from this system. Enhanced or constitutive secretion of siderophores could shed light on the possible upper limit of siderophore-mediated silicate dissolution, and contribute to the broader understanding of engineered synthesis and production of complex natural products. In some cases, siderophores are not a chemical monolith, but rather a functional category composed of highly diverse chemical structures and backbones, which influences their bioavailability, chemical behavior in water, and photoreactivity. Exploring the specific interactions between siderophores and olivine will better inform possible designer siderophores that are both metabolically cheap to produce, and optimized for catalyzing dissolution.

Genetic engineering may also enhance other mechanisms of mineral dissolution, such as biofilm formation, citrate production, and local acidification. Biofilm formation in the context of pathogenesis, both genetically and physiologically, may result in a range of therapeutics and drugs that disrupt biofilm formation. Biofilms could be utilized industrially for olivine dissolution and other applications such as microbe-electrode interactions, but engineering and controls of film formation and architecture remains difficult.

Many of the phenotypes described here, such as siderophore production and biofilm formation are difficult to engineer rationally due to their complex genetics. In the system proposed here, however, microbial growth may be coupled to the harvesting of iron from olivine and other silicate minerals. It may be possible, therefore, to perform large scale selection of mutants for enhanced harvesting of nutrients from minerals. High throughput selections or screens could reveal key targets for rational genetic engineering, and identify high-performing gain-of-function mutations that have immediate industrial utility.

Bottom-up genetic engineering and synthetic biology can improve performance of co-culture or consortia systems. For operation at large scales, stability of a multi-component living system is challenging, especially in complex, non-sterile mediums such as seawater. Mutual dependencies between microbial organisms have been extensively described in natural systems, and applied to synthetic systems at lab scale. To date, synthetic microbial consortia have not been implemented at industrial scale, however. The olivine dissolution system described here, specifically, offers a novel solution for synthetic mutual dependency.

Finally, metabolic engineering and synthetic biology have been extensively applied to synthesize valuable biomolecules at high titers in engineered hosts. These include biomass components such as lipids, complex natural products, biofuels, and medicines. Similar techniques are applied to enhance the growth of microorganisms on renewable feedstocks, such as lignocellulose, or plastic polymers as discussed earlier. One additional microbial application of olivine dissolution is the uptake and concentration of valuable trace metals. Metal uptake by bacteria has been studied in the context of heavy metal remediation and plant-microbe interactions. A system for microbial dissolution of minerals provides a uniquely useful platform with which to study the uptake and harvesting of valuable metals.

Utilization of genetically engineered organisms for large-scale mineral dissolution requires consideration of the ethical and ecological impacts. One advantage of the proposed system is the potential for physical containment on top of genetic controls. Furthermore, engineering controls may protect against unwanted mutation or ecosystem proliferation. Recently reported organisms with a reduced genetic code may be less likely to acquire natural genetic material that could confer new phenotypes and contribute synthetic genetic material to the natural gene pool. Novel kill-switches and synthetic auxotrophy may also restrict activity and replication of engineered organisms to controlled environments. Early examples of deployment of engineered organisms such as engineered crops with resistance to pests or drought provide a framework within which new technologies may be evaluated.

In short, physical, chemical, and genetic conditions may be engineered to optimize metabolic functions, as well as the thermodynamics and kinetics of mineral dissolution and carbon capture. Such cultivation systems can be operated within a broader existing economic and industrial infrastructure, where they can act not only as systems of large-scale carbon capture, but also offer sustainable means of waste management and/or production of valuable by-products.

Considerations for Mineral Feedstocks and Industrial Cultivation

Large-scale (Mt/Gt) CDR reactors will require large inputs of water, nutrients, and mineral feedstock, and will require downstream processing to recover valuable added products. Industrial infrastructure and scientific knowledge already exist to implement such reactors at scale: combined, the mining and construction sectors produce >8 Gt/year of mineral waste materials with CDR potential>8 Gt/year. The remaining questions then relate to finding the upper limits of dissolution and carbon capture rates that such reactors can offer, while identifying the operating conditions that will sustain these at scale.

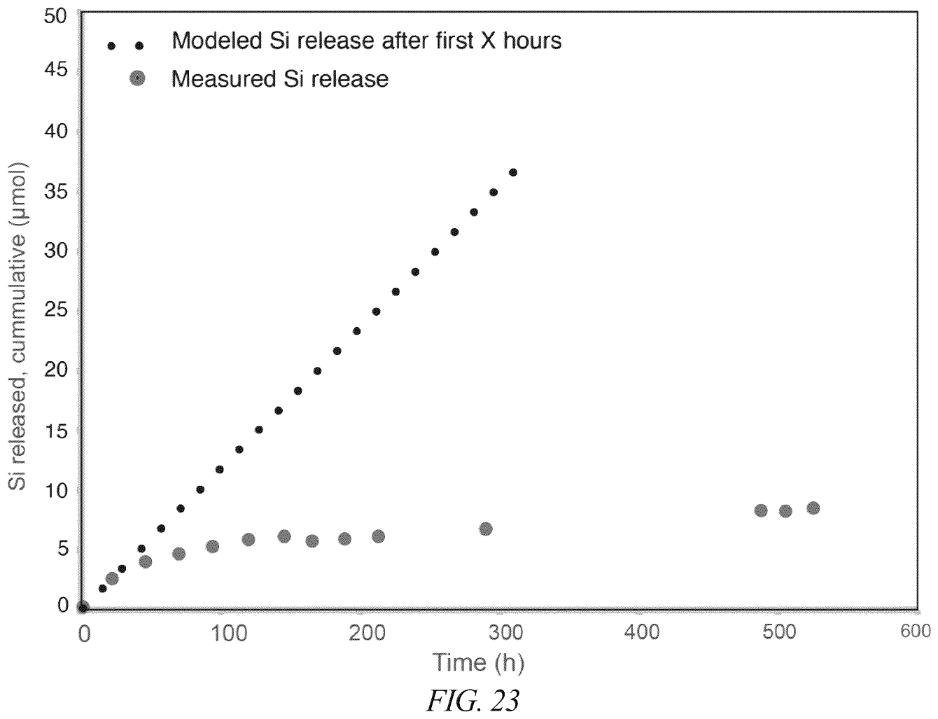

Some of the key questions that remain include identifying the conditions for maximal mineral dissolution and carbon capture rates. Reaching these maxima will involve experimenting with a wide range of physical and chemical constraints, as these help manage the baseline thermodynamic environment of mineral dissolution. As discussed, silicate dissolution rates can be difficult to constrain over periods of years. There is a significant bias in the literature, where quantified measures of dissolution have focused on short time periods of hours-days, which are not representative of long-term steady-state dissolution rates. FIG. 23 shows a modeled dissolution rate derived from the first several hours of dissolution versus empirical dissolution data over several weeks. Characterizing mineral dissolution over months can however be challenging, as dissolution can be non-stoichiometric, and different proxies (i.e., dissolved silicon vs. nickel) can give very different quantified estimates of dissolution rates.

Additionally, a comprehensive characterization of mineral substrates and their geochemistry is key. As discussed, a mineral's dissolution rate and potential to generate alkalinity are critical characteristics in determining their carbon capture potential. In this respect, ultramafic minerals such as olivine and serpentine are of great interest. However, associated reactions can negatively impact carbon capture efficiency, principally among them the oxidation of iron. As a major component of olivine and many ultramafic minerals, ferrous iron will oxidize readily during mineral dissolution, a reaction that releases free protons and neutralizes the alkalinity generation congruent to silicate dissolution. Choosing ultramafic minerals with a low iron content would be important in abiotic conditions. However, in the context of bioreactors that promote microbial iron acquisition, it is unclear what iron content will maximize dissolution rates. Furthermore, the processes involved in the microbial acquisition of iron (i.e., ligand exchange reactions between siderophores and ferric iron, ferric reduction involved in iron cellular absorption, along with photoreduction of ligand-bound iron), are all proton-sensitive reactions, and can impact pH, and thus alkalinity generation to some extent. Characterizing this process will help constrain system design for maximal carbon capture.

Another critical component in estimating net CO₂ fluxes relates to the fate of carbon held in the organic pool. Characterizing the fluxes and fate of carbon across processes like biological assimilation, biofilms, mineral dissolution, and secondary precipitation in a dense environment is a challenging yet exciting and meaningful scientific endeavor. However, for the purposes of carbon capture, keeping track of total carbon fluxes in and out of the system, and net carbon capture, is a much more straightforward effort. While the single most important metric for a bioreactor might be net carbon fluxes and associated carbon capture rates, it will be important to characterize the fate of carbon and all its forms. This implies steep geochemical and metabolic gradients, forcing a wide range of reactions that can impact the form and fate of carbon. The main forms of carbon flowing out of bioreactors will be dissolved as well as particulate organic and inorganic phases. Being able to account for all their relative abundances will be valuable, not only as an additional means to measure carbon capture and potentially useful biomolecule production, but also to inform on strategies around reactor design, engineering and permanent carbon storage.

Mg2-xFexSiO4+4CO2+4H2O-->2-xMg2++xFe2++4HCO3-+H4SiO4 [Eq. 1]

Inorganic carbon fluxes are highly dependent on a system's carbonate saturation and precipitation as secondary mineral phases. As per Eq. 1, the most efficient form of carbon capture is in the form of bicarbonate (also referred to as dissolution trapping). An ideal bioreactor system should therefore maintain conditions below carbonate and silicate saturation in order to limit the precipitation of secondary minerals. Although the precipitation of carbonates still produces a net sink of stored carbon (also referred to as mineral trapping, or carbonation), the acidity simultaneously generated in this reaction decreases the efficacy of the process significantly. The flexibility of an engineered system offers the possibility to promote and effectively manage carbonate precipitation as a form of long-term storage.

Hence, a comprehensive characterization of accelerated mineral dissolution geochemistry under a wide range of conditions will be critical to maximize carbon capture, and the forms of carbon must be fully characterized in order to best manage its permanent storage. Carbonate precipitates can be easy to store permanently since they are relatively unreactive in the natural environment. Bicarbonate and DIC however need to be stored in vast, well buffered basins such as the world's oceans in order to remain stable and not outgas CO₂ back into the atmosphere. Organic forms of carbon (particulate and dissolved) will need to be stored in an environment where it will avoid reoxidation, which might involve specific containment strategies.

### Techno-Economic Viability

The total CDR potential of known, global reserves of ultramafic minerals lies in the range of several tens of thousands of gigatons of CO₂, orders of magnitude greater than what is required to sequester anthropogenic CO₂ emissions. While geochemical CDR thus offers tremendous potential, implementing it at scale carries economic constraints that must be taken into account. Techno-economic analyses of large-scale reactors are critical in guiding research and development, highlighting constraints that are not always obvious in a lab or small scale setting.

By estimating the dimensions and requirements of a reactor at scale, one can infer the costs and logistical constraints of such an operation. Namely, these will include the costs of building the reactors, excavating and grinding the ultramafic minerals, the costs of fertilizers and other substrates, accessing and treating the water used in reactors, as well as total energy requirements. The economic potential of carbon capture also offers a unique opportunity for industries that utilize microbial catalysis as a process of chemical engineering. Some industries have been extremely successful (i.e., fermentation and the alcoholic beverages industry), and some have struggled to remain competitive (i.e., algae farms and biofuels industry). Carbon capture reactors, however, sell, at their core, carbon credits. The extraction of valuable byproducts from these bioreactors, such as metals (e.g., nickel or cobalt), or organics (e.g., lipids or vitamins), does not need to be a profitable process in of itself, since the main operational goals will be to capture carbon at an efficient cost. In fact, the recovery of valuable byproducts will serve to subsidize the cost of carbon capture, and will further help integrate CDR efforts into the broader economy, by providing a source of valuable commodities to other sectors including agri- and aquaculture, pharmaceuticals, and industries. This represents an unprecedented opportunity to connect the accelerated weathering of the ultramafic minerals to rapidly growing carbon markets projected to surpass $1 Trillion in 2050, as net-zero pledges from countries and companies continue to skyrocket.

Thus far, our techno-economic analyses show that industrial-scale bioreactor systems for enhanced weathering are comparable to common water treatment facilities, and can thus benefit from existing infrastructure and engineering capabilities. With the scientific proof of concept already established, our analyses indicate that methodological and technological improvements to scale bioreactors as described above has the potential to achieve net cost of carbon capture (including capex, opex, energy and carbon costs, substrate inputs, etc.) between −$20/tonne and $100/tonne. The negative dollar value per tonne derives from the potential to subsidize carbon credit costs through the recovery of diverse value added products. Because of these products, this process not only has the potential to be inherently profitable, but could be considered a simultaneous carbon capture and mining and resource extraction process.

### CONCLUSION

Due to inaction for broad decarbonization coupled to continuous anthropogenic greenhouse gas emissions, there is a global consensus that large-scale CDR will be needed to remove gigatonnes of CO₂ over the next several decades. CDR will also be necessary to remove further CO₂ emissions required for the transition to a decarbonized, green economy. Accelerating Earth's inorganic carbon cycle through the weathering of silicate minerals like olivine offers a promising path to removing and storing gigatonnes of atmospheric CO₂. Due to the sheer amount of CO₂ that needs to be sequestered, multiple weathering approaches must be pursued simultaneously. While grinding minerals into sand-sized particles can accelerate weathering rates of olivine minerals in coastal environments (i.e. Coastal Enhanced Weathering), half lives of olivine grains are on the order of decades. Hence, we suggest that microbial catalysis to accelerate mineral dissolution rates must be explored alongside other CDR methods. We have shown that microbes have the potential to significantly increase weathering rates leading to olivine grain half-lives of several years. By utilizing micronutrients like Fe in olivine, microbes can use these mineral substrates for exponential growth, thereby offering a path to scale the cultivation of microbiomes growing on silicate minerals. Indeed, other industries have successfully cultivated microbes to perform specific, valuable functions including wastewater treatment and fermentation. Further support to examine the biological mechanisms of microbial-mineral interactions, biofilm formation, and large-scale cultivation will provide critical insights that can galvanize global efforts in silicate dissolution for CDR. It will also create a step-change in our understanding of fundamental microbial processes particularly in biofilm formation and the synthesis of complex natural products, while also opening the door to novel approaches to engineer microbiomes. These research endeavors naturally integrate the fields of CDR, synthetic biology, geochemistry, climate science, engineering, and economics and will provide new models that can yield various outputs from the sustainable biosynthesis of valuable products to the removal and long-term storage of excess atmospheric CO₂. While many of these fundamental processes have been happening worldwide for billions of years, we are only taking the beginning steps along the pathway towards understanding how these mechanisms can help turn the tide on the climate crisis.

### System Design

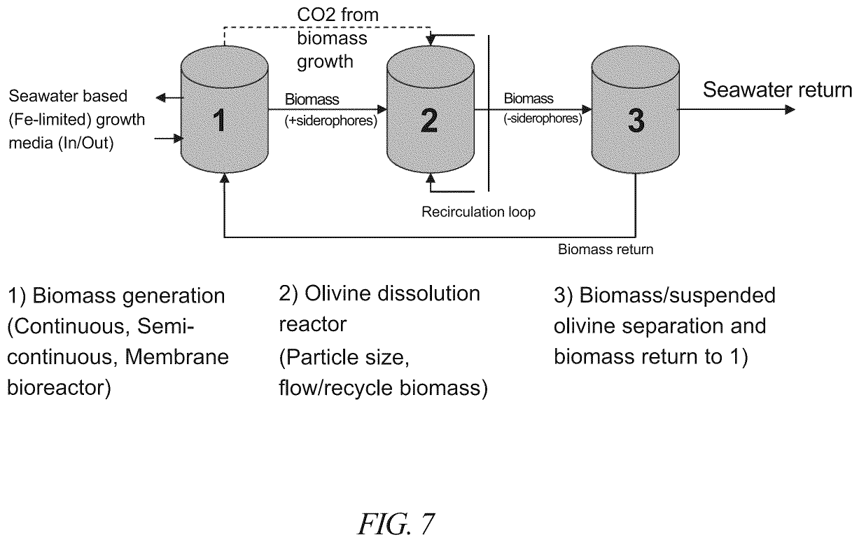

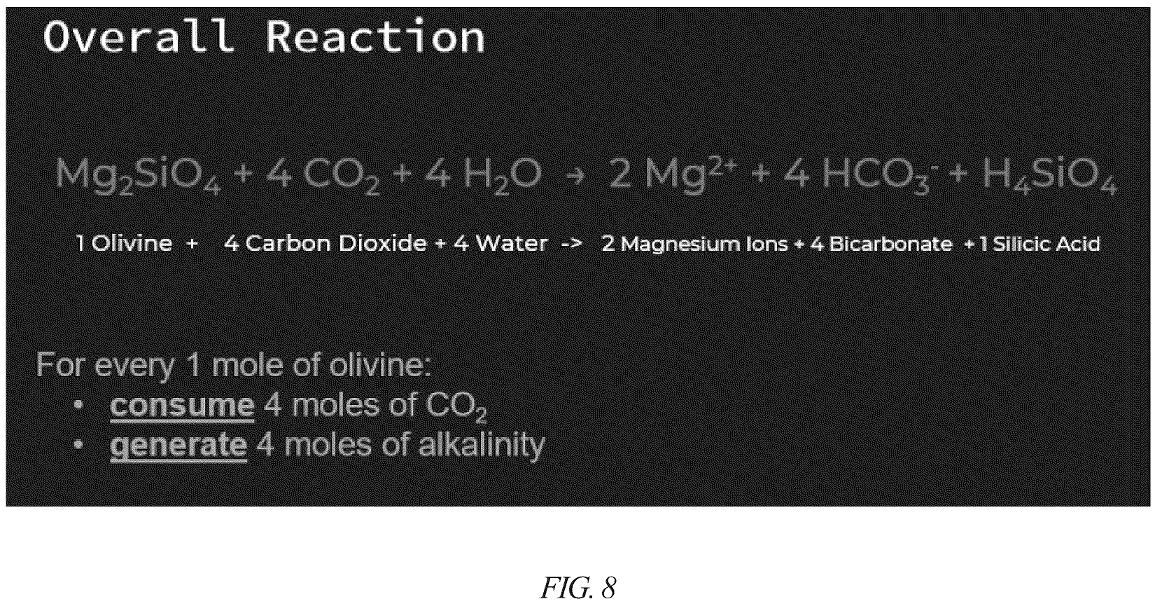

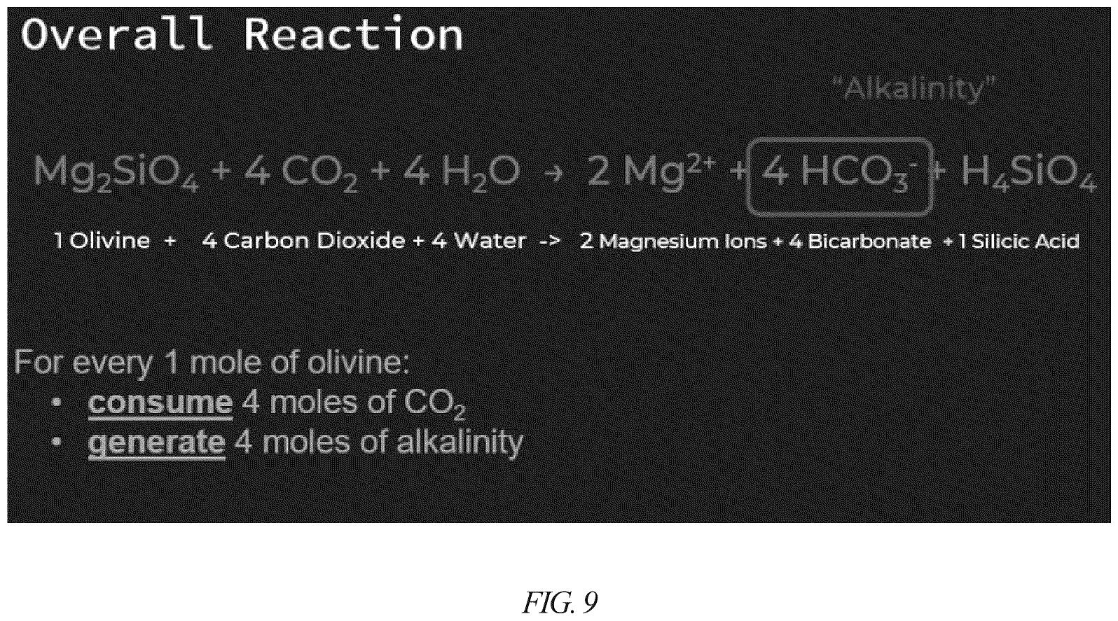

Referring to FIG. 7, the illustrated reactor may be configured to intake seawater that may be mixed with olivine and microbial biomass to generate alkalinity. Depending on the source, seawater may have to be pretreated prior to reacting with olivine. This reaction may also result in trace metals, microbial biomass, and other reaction products mixing in with the seawater medium. In order to sequester carbon at scale, this alkaline seawater may be returned to the ocean to induce atmospheric carbon dioxide uptake. Importantly, the return of this seawater containing the certain constituents must meet local regulations. Another microbially-mediated treatment process that returns effluent to the environment may involve anaerobic digestion. This may be a good reference to understand regulations for effluent from microbial processes.

In some non-limiting embodiments, one or more stirrers may be used to facilitate the carbon capture process (e.g., by physically mixing the seawater containing olivine sediments with the medium comprising the carbon dioxide). The medium may comprise a liquid medium (e.g., seawater) or a gaseous medium (e.g., air comprising atmospheric carbon dioxide). In some non-limiting embodiments, one or more valves may be used to control and regulate an outflow of any products of the reaction (e.g., the magnesium, bicarbonate, and/or silicic acid).

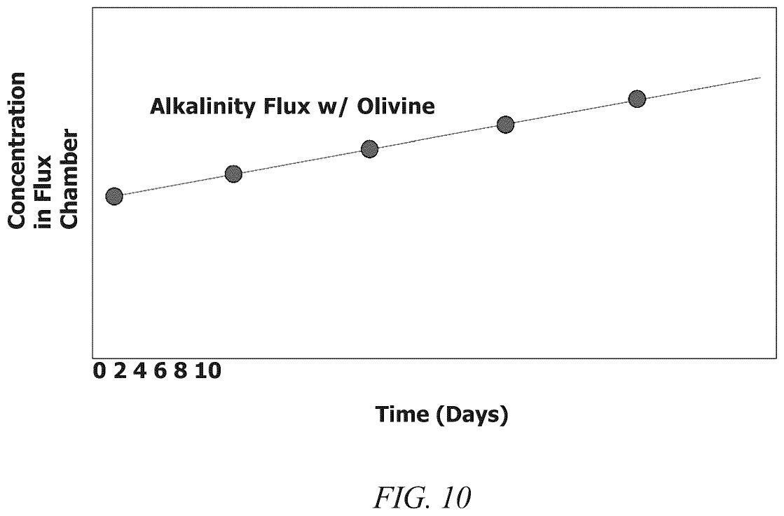

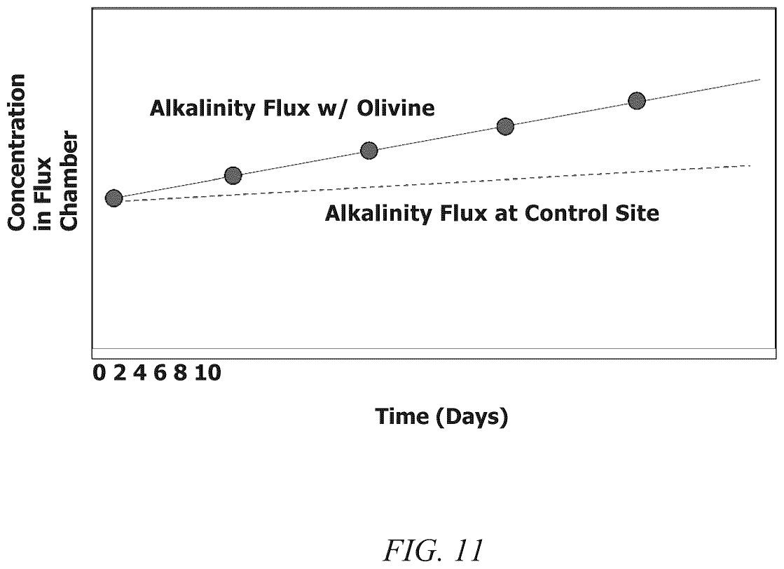

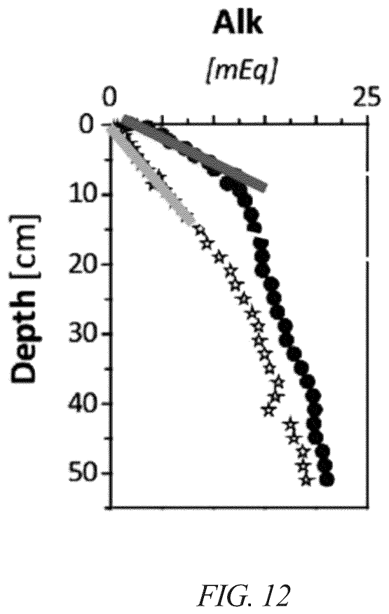

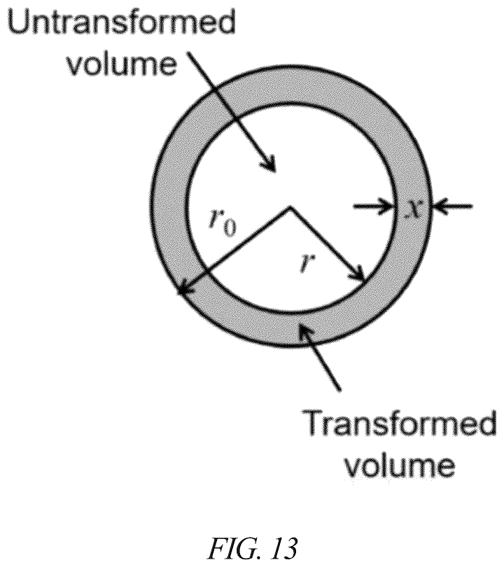

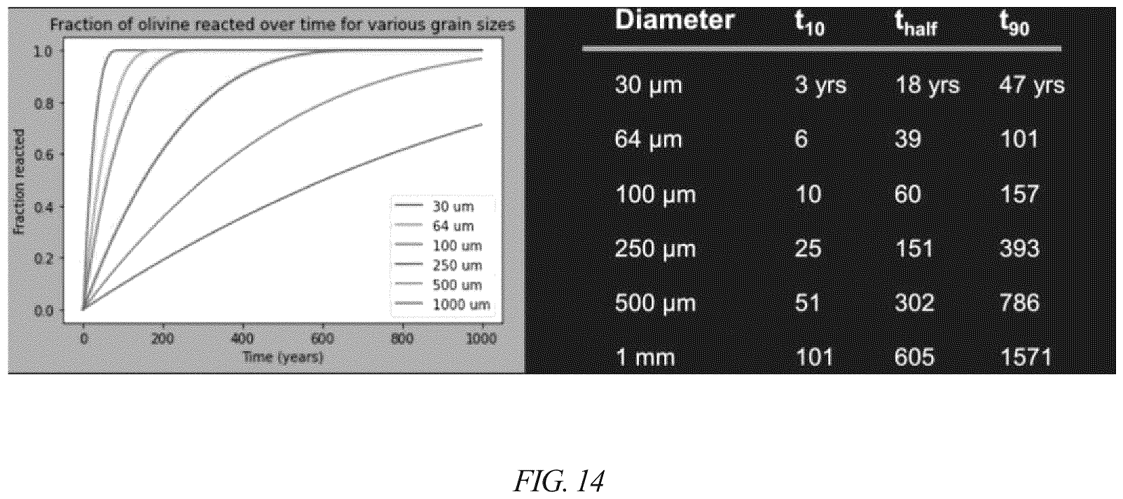

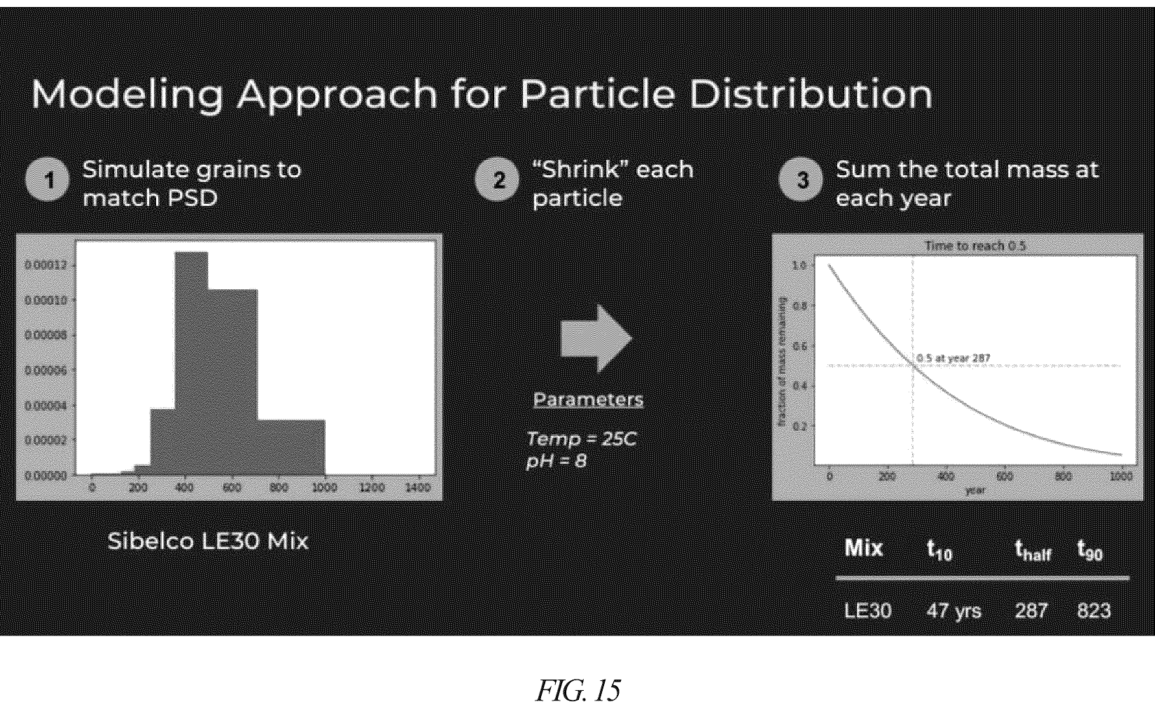

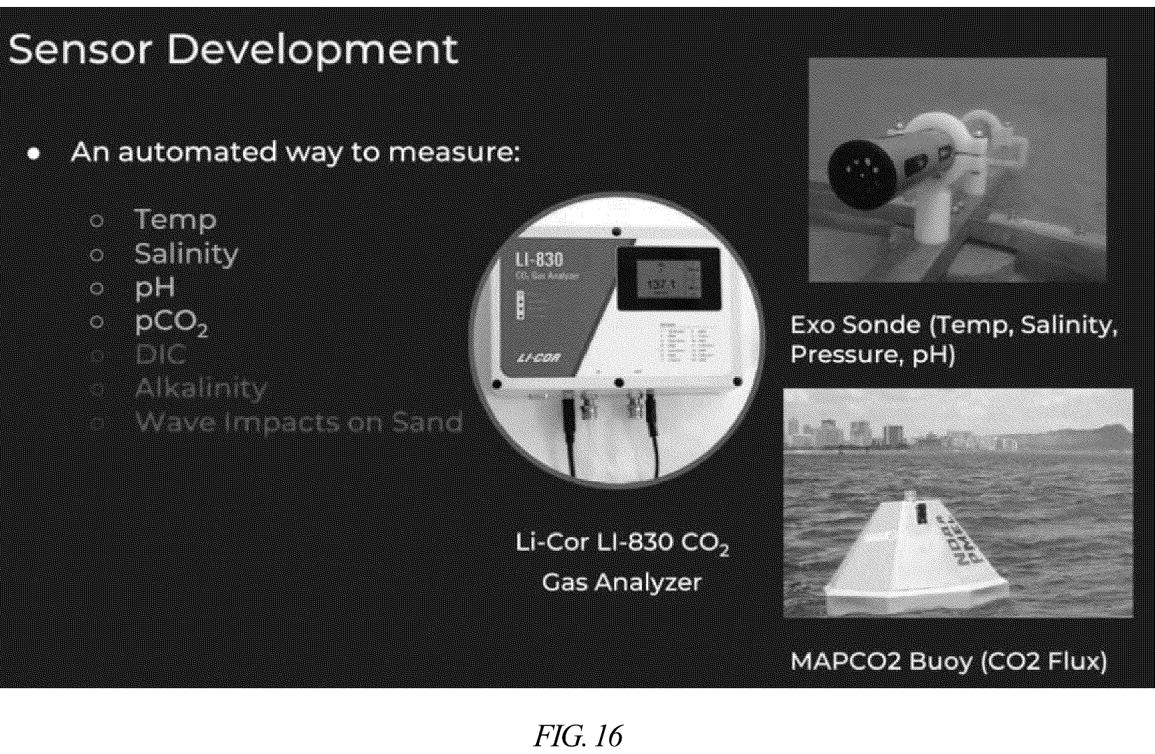

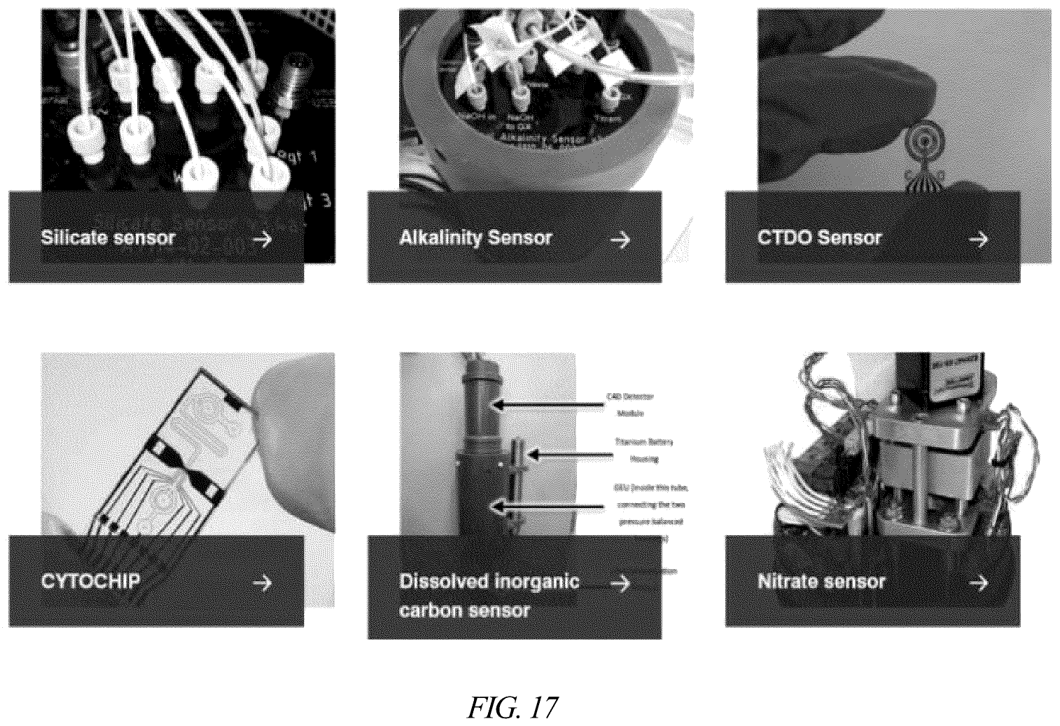

FIG. 10 illustrates a plot of a change in concentration of an alkaline material in a flux chamber as a function of time when the olivine-based carbon capture reactions of the present disclosure are used. The alkalinity of the medium in which the alkaline material is present may gradually increase over time. In some cases, the alkalinity may increase by a factor of 1.1 to 10 or more over a period of 1 to 10 days. As shown in FIG. 11, the alkalinity flux with olivine increases more rapidly over time than the alkalinity flux at a control site in which olivine is not introduced. The alkalinity flux may correspond to the slope of the lines shown in these plots, and may have units of change in concentration of the alkaline material per unit time.

In some cases, the reactor may comprise a first chamber for biomass generation (e.g., continuous, semi-continuous, membrane bioreactor), a second chamber for olivine dissolution (e.g., to optimize particle size and flow/recycle biomass), and a third chamber for biomass/suspended olivine separation (and for biomass return to the first chamber). In some cases, sea water based growth media may flow into and/or out of the first chamber. In some cases, carbon dioxide from biomass growth may be provided to the second chamber. The second chamber may also receive biomass with siderophores from the first chamber. The second chamber may provide, in some cases, a recirculation loop for the biomass. The second chamber may be configured to provide the biomass without the siderophores to the third chamber. In some cases, the third chamber may be configured to return biomass to the first chamber. In some cases, the third chamber may be configured to provide the olivine and/or the biomass to seawater. In some cases, the third chamber may be configured to enable Technology Metal extraction. In some cases, the third chamber may flow water to an additional chamber to recover Technology Metals.

The present disclosure effectively provides methods and designs for sustained microbial growth and siderophore production for olivine dissolution over long time periods. Current methods are unable to accelerate olivine dissolution with microbes for reaction volumes exceeding a few hundred milliliters, unlike the systems and methods disclosed herein. The presently disclosed systems and methods have been developed with the specific engineering and regulatory requirements needed to build large-scale, flow-through reactors with seawater that will then be returned to the local environment. The present disclosure effectively provides systems and methods for optimizing (1) strain selection for olivine dissolution, (2) grain size, (3) culturing conditions with natural seawater, and (4) the materials for the growth chamber.

The systems and methods of the present disclosure may be implemented to optimize dissolution rates by (1) inducing or imparting wave energy with a specific characteristic (e.g., periodicity, intensity, motion path, etc.) and/or (2) modifying the biotic processes used to enhance olivine dissolution.

### Method of Removing Atmospheric Carbon

Bioreactors with olivine sand can be used to accelerate Earth's natural carbon dioxide removal process. At current rates, the natural process of rock weathering through rainfall needs to be sped up by at least 100 times to absorb the carbon dioxide emitted by human activity.

### Mechanics and Chemistry of Weathering and Dissolution

Using microbial processes to accelerate the dissolution of carbon-removing sand is critical to the efficiency of carbon dioxide removal. Past analyses have shown that grinding carbon-removing sand to <100 μm size is highly energy-intensive. However, grinding carbon-removing sand to >300 μm sand requires far less energy. In some cases, the olivine material within the bioreactor has a particle size that is less than or equal to about 10 mm, 5 mm, 2 mm, 1 mm, 900 μm, 750 μm, 600 μm, 500 μm, 450 μm, 400 μm, 350 μm, 300 μm, 250 μm, 200 μm, 150 μm, 100 μm, 50 μm, 25 μm, or less. In some cases, the olivine material within the bioreactor has a particle size that is greater than or equal to about 25 μm, 50 μm, 100 μm, 150 μm, 200 μm, 250 μm, 300 μm, 350 μm, 400 μm, 450 μm, 500 μm, 600 μm, 750 μm, 900 μm, 1 mm, 5 mm, 10 mm, or greater. In some cases, the olivine material within the bioreactor has a particle size that is between two values described above, for example between about 250 μm and about 500 μm.

In some cases, grain-on-grain collisions within the bioreactor may further accelerate the breakdown of olivine materials (e.g., carbon-removing sand). Carbon-removing sand grains may be reduced in size due to mechanical activation. In some cases, the motion of the water in which the carbon-removing sand is provided may cause surface abrasions. In some cases, the surface of the carbon-removing sand grains may be mechanically activated to enable and/or enhance carbon dioxide uptake and de-acidification.

### Dissolution Kinetics

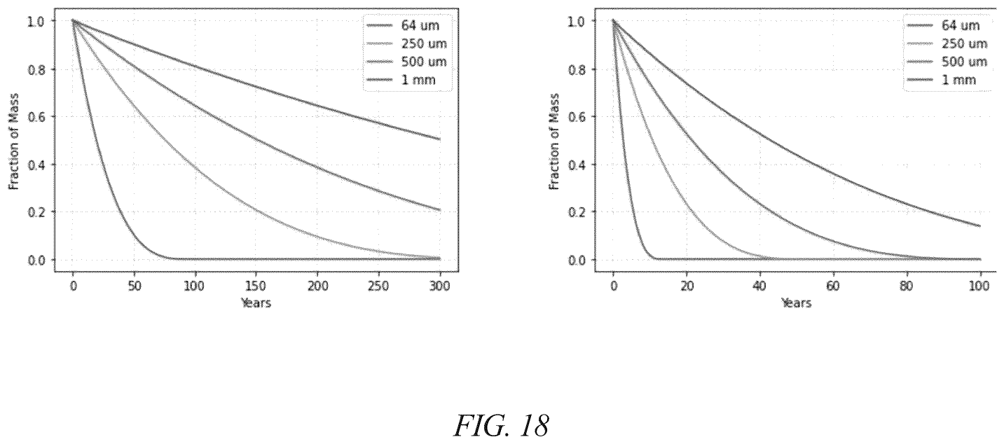

FIG. 18 illustrates various plots showing exemplary olivine dissolution kinetics for olivine particles having different grain sizes. Dissolution time may decrease as carbon-removing sand particle sizes decrease. The particle dissolution characteristics may be predicted or simulated using a shrinking core model and one or more laws, equations, or principles governing carbon-removing sand dissolution rates. In some cases, the shrinking core model may be modified to permit the simulation of dissolution for realistic, commercially-available grain size distributions of carbon-removing sand. The models described herein may be generated by modifying initial diagenesis models to incorporate carbon-removing sand dissolution kinetics that correspond to the shrinking core model. The models may include and/or account for full carbonate chemistry (e.g., DIC, ALK, pCO₂, pH, carbonate precipitation/dissolution) as well as biogeochemical processes (including aerobic and anaerobic respiration, formation of Fe/Mn oxides, pyrite, sulfide oxidation, etc).

### Secondary Minerals

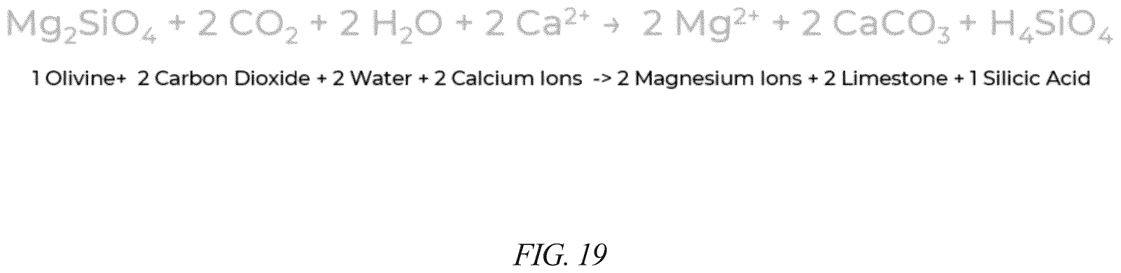

In some instances, secondary carbonate precipitation as shown in FIG. 19 can impact carbon dioxide capture efficiency. Secondary carbonate precipitation may change the reactions described herein such that for every 1 mol of olivine introduced to a target environment, 2 moles of carbon dioxide are consumed and 0 moles of alkalinity are generated. This can result in a 50% reduction in carbon dioxide capture efficiency. The systems and methods disclosed herein may be implemented to minimize or reduce secondary carbonate precipitation. The rate of carbonate precipitation can be strongly controlled by local variations in geochemistry and biogenic calcification. In some cases, alkalinity export can be the dominant process but secondary minerals can form depending on the saturation state of calcium carbonate in the local seawater. Bioreactor conditions can be modified to minimize or reduce the formation of secondary carbonates. For example, air mixtures with elevated carbon dioxide concentrations can be bubbled into the seawater medium to reduce the potential formation of secondary carbonates. The secondary carbonate precipitation may be a solid-state carbonate, which causes carbon to be stored as a solid carbonate mineral.

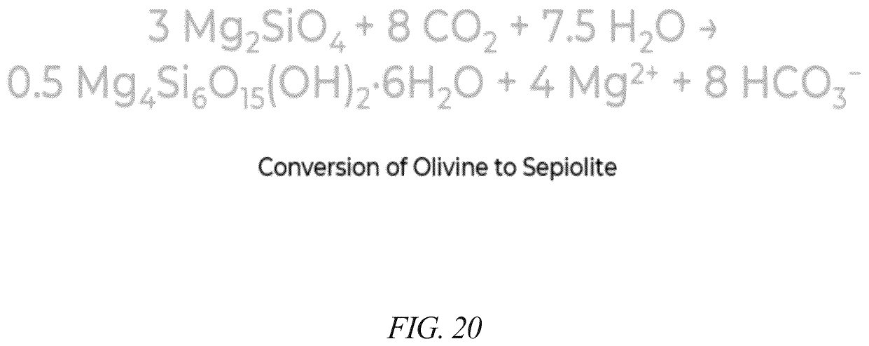

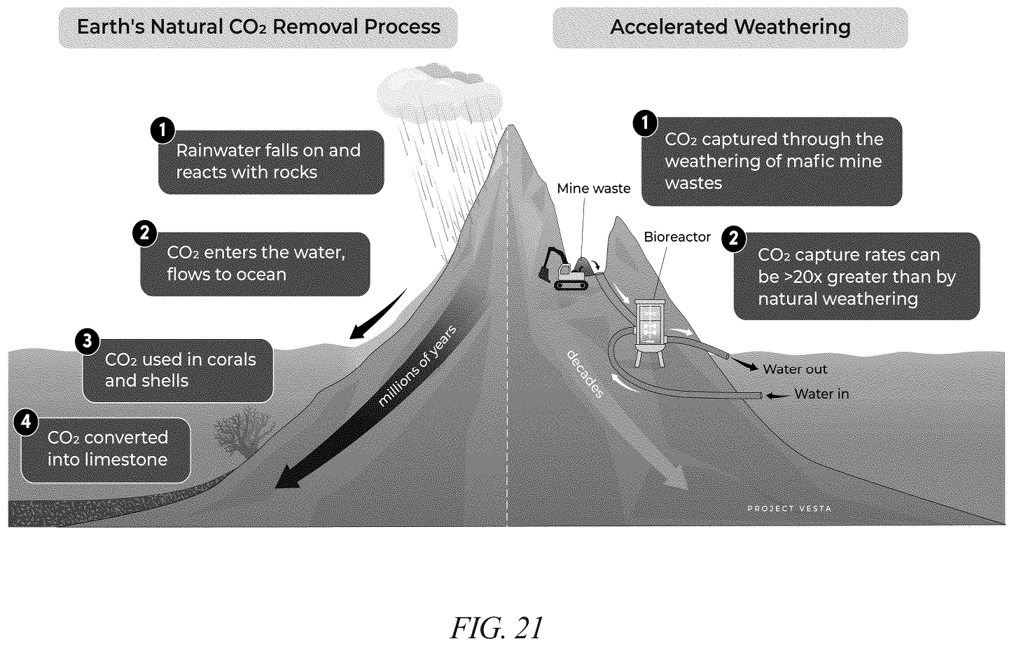

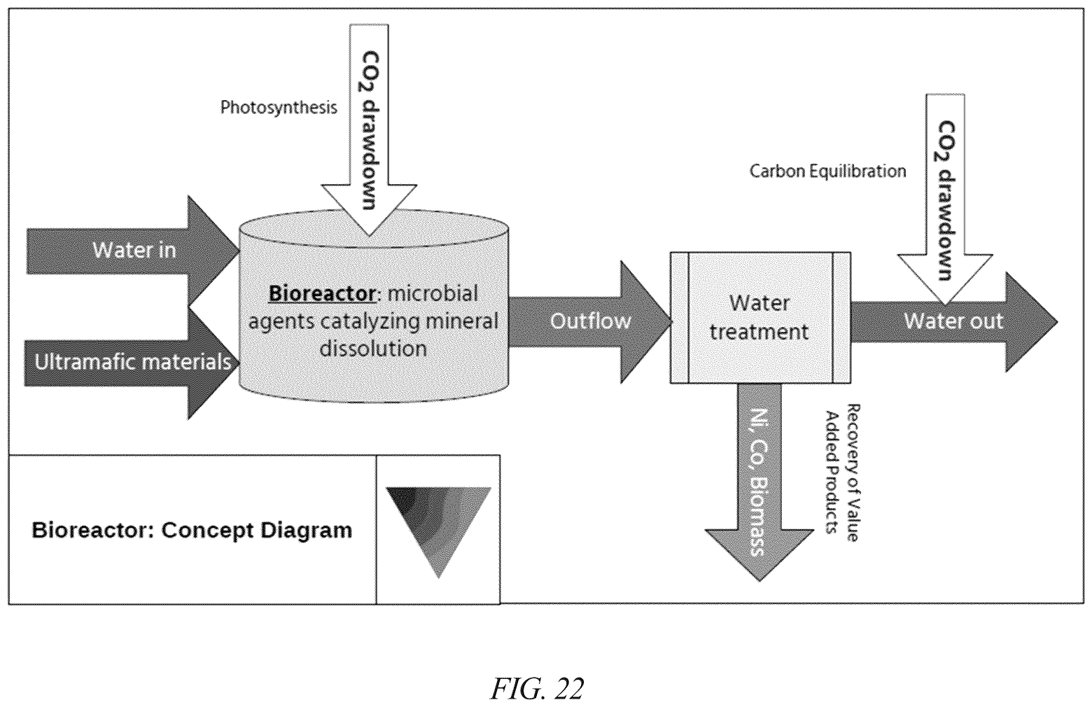

In some instances, secondary clay formation as shown in FIG. 20 can impact carbon dioxide capture efficiency. Secondary clay formation may change the reactions described herein such that for every 1 mol of olivine, 8/3 moles of carbon dioxide are consumed and 8/3 moles of alkalinity are generated. This can result in a 33% reduction in carbon dioxide capture efficiency. The systems and methods disclosed herein may be implemented to minimize or reduce such secondary clay formation.

The present disclosure also provides software configured to calculate carbon dioxide consumption and to measure, record, and verify carbon removal. The software may be configured to determine carbon dioxide consumption and/or carbon removal based on one or more sensor readings, model outputs, and/or one or more input parameters. The one or more input parameters may relate to, for example, the physical or chemical properties of the carbon-removing sand used, the ratio of carbon-removing sand to microbial biomass, the physical and chemical properties of the bioreactor seawater, the ratio of carbon-removing sand to siderophore concentrations, or the manner/configuration in which the carbon-removing sand and microbes are suspended (e.g., method of mixing within the reactor). In some cases, the sensor readings may comprise measurements of alkalinity, DIC, pCO₂, pH, salinity, conductivity, dissolved oxygen, nutrients, trace metals, organic carbon, water temperature, agitation, and/or additional chemical and physical properties. Data from any individual or combination of sensors described herein may be used to calculate carbon dioxide consumption, conditions of the bioreactor, or other performance metrics.

In one aspect of the present disclosure, systems and methods of microbially-driven geochemical negative emission technologies (NETs) are described. Microbially driven NETS may be defined by comprising one or more biochemical agents as an element of their Carbon Dioxide Removal (CDR) process. These biochemical agents may include bacteria, archeaea, sub-millimeter-scale eukaryotes, viruses, exometabolites, or extra-cellular enzymes and nucleic acids. In some cases, CDR may be performed by metabolic activity or biochemical activity of these biochemical agents.

A system configured to run a NET may comprise a biochemical process configured to catalyze CDR. In some cases, the biochemical process may be one or more microbial or enzymatic processes configured to accelerate the drawdown and incorporation of carbon dioxide into a storable form. A non-limiting example of a storable form of carbon dioxide includes carbonates. In some cases, the biochemical process may comprise using electromagnetic or chemical energy to capture carbon dioxide and transform it into organic carbon. Non-limiting examples of a biochemical process include the Calvin cycle or Wood-Ljungdahl cycle. In some cases, the biochemical process comprises biosynthesis of metabolites, such as ligands, extracellular polymeric substances, or organic acids. These metabolites can directly or indirectly catalyze mineral dissolution. These metabolites may be produced in a planktonic environment within sediment porewaters, or within the context of biofilms. FIG. 1 shoes an example of this process and its effectiveness. FIG. 1 shows the enhanced release of nickel and olivine dissolution rates as a function of microbial exometabolites activity (top line) as compared to abiotic conditions (bottom line). In some cases, biochemical processes comprise one or more processes that promotes the nucleation and precipitation of solids, (e.g., mineral or amorphous phases) that act as a sink for carbon. Examples of such solids include carbonate or silicate solids.

In another aspect of the present disclosure, embodiments of microbiomes are described. In some cases, a microbiome may comprise a plurality of bacteria species. One or more bacteria of the plurality of bacteria within the consortium may be genetically engineered. One or more species of bacteria of the plurality of bacteria within the consortium may not be genetically engineered. In some cases, providing the consortium of bacteria may include designing the type of bacteria within the plurality. In addition, providing the consortium may comprise providing specific species of bacteria, genetically engineered or not, in certain amounts or ratios to generate a resulting consortium where individual species of bacteria within the consortium add synergistic effects to other species of bacteria and result in a net increase level of activity of olivine weathering, carbon dioxide removal, or other biological metabolic activities described herein.

In another aspect, the present disclosure provides methods of modifying or controlling the viability and composition of a consortium of bacteria described herein. Controlling physical and chemical environments of the consortium of bacteria (e.g., within a reactor, chamber, or reservoir of a system described herein) may be conducted to promote specific metabolisms and pathways. In some cases, such controls may comprise engineering genomes of individual species (e.g., strains) of bacteria through synthetic biological methods to create metabolic co-dependencies between two or more species (e.g., strains) of bacteria of the consortium.

In another aspect of the present disclosure, certain methods for process engineering of carbon removal processes are described. For example, in addition to tools of biochemistry and microbiology, microbially-driven geochemical NETs can leverage engineering tools to enhance CDR by controlling and optimizing chemical and physical conditions to meet desired objectives. In one aspect, the effectiveness of biochemical agents can be significantly impacted by the physical and chemical environment in which they operate. Factors of the physical and chemical environment include, but are not limited to, nutrients, gases, mineral substrates, temperature, light, and water fluxes. In some cases, one or more of these factors are controlled, monitored or provided to increase an effectiveness of a biological process. Nutrients as described herein may include organic or inorganic nutrients such as nitrogen, phosphorous, silicon, and carbon, as well as metals such as zinc, copper, and tungsten. One or more of these nutrients may be provided to an environment (e.g., deployment site, reactor, etc.) of a biochemical agent. Another factor to control a biological activity described herein may be the control, addition, or removal of gases (e.g., carbon dioxide, oxygen, and hydrogen) from an operational environment. In some cases, one or more gases may be provided to an operational environment by injection of the gas(es) into the environment medium, or by introducing chemically reactive materials that generate the gas(es) under conditions of the environment.

A variety of mineral substrates can be utilized as a material for enhanced weathering. These generally involve mafic and ultramafic materials and salts, including but not limited to, dunite, basalt, or brucite, and can be acquired from a variety of sources, including but not limited to new materials freshly extracted from the ground, or waste products from industries such as mining or smelting. These substrates can be introduced into the microbially-driven geochemical NETs under a range of grain sizes (e.g., clay-sized to pebble-sized), crystal structure (e.g., amorphous, glass or crystalline), or packaging forms (e.g., pellets).

Capturing large amounts of CO₂, along with dissolving large quantities of minerals and generating large quantities of biomass creates an opportunity in the production of value-added products, but also a need to manage the forms of carbon for permanent storage, as well as means to protect the environment form undesirable impacts.

The large-scale dissolution of minerals can lead to the release of large quantities of valuable metals such as nickel. Similarly, the large amounts of biomass production can lead to the synthesis of valuable organic compounds, such as specific lipids, vitamins, or ligands. The recovery of these valuable commodities can be critical in implementing cost-efficient large-scale operations. Microbially-driven geochemical NETs can implement methods of value-added byproducts by known methods such as, though not limited to, adsorption matrices (e.g., resins, metal oxides, etc.), or the use of solvents (e.g., Blight & Dyer process).

In order to protect a natural environment from undesired biochemical agents, methods and systems described herein (e.g., microbially-driven geochemical NETs) can implement treatment methods onto its effluents, such as though not limited to concentrated UV treatment, or genome-based self-lysing systems.

Recovering and storing various forms of carbon, particularly solid forms such as minerals (e.g., carbonates) or organic (e.g., biomass), may be critical in order to achieve permanent storage. To this effect, microbially-driven geochemical NETs may have to recover and separate these forms instead of releasing them to the environment. This can be performed by known methods such as, though not limited to, centrifugation, flotation, or filtration.

In some instances, one or more sensors may be disposed in, around, or on the bioreactor. Sensors may be deployed within, on or around one or more chambers or fluidic passageways of the bioreactor.

In one aspect, the present disclosure provides methods and protocols for measuring, recording, and verifying (MRV) carbon removal (e.g., by way of bioreactors using carbon-removing sand). The protocols may be submitted to independent, third-party entities (e.g., academics, institutions, etc.) for validation. Once validated, the protocols may be implemented by an individual or an entity. Additional third-party entities may ensure and confirm compliance. The individuals or entities implementing the MRV protocols may then submit the third-party verified, compliant methodologies to a carbon credit verifier, and an offset or a credit may be issued on a registry for sale in domestic and/or international markets.

### Computer Systems

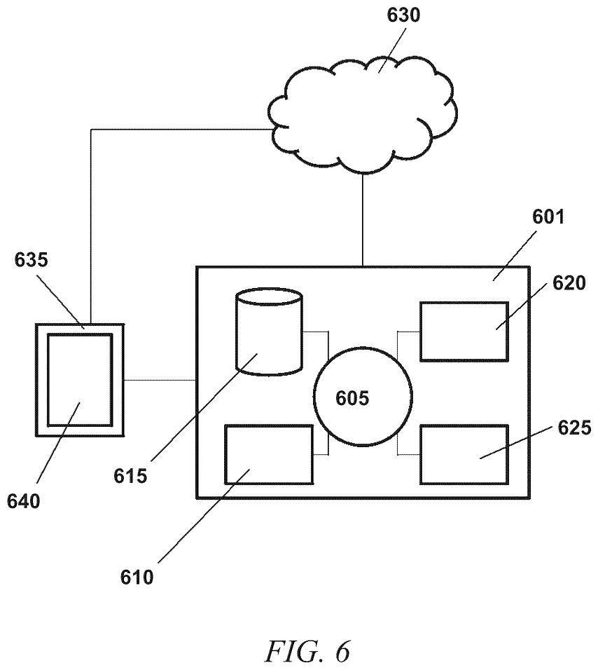

In an aspect, the present disclosure provides computer systems that are programmed or otherwise configured to implement methods of the disclosure, e.g., any of the subject methods for processing and distributing olivine. FIG. 6 shows a computer system 601 that is programmed or otherwise configured to implement a method for olivine processing and distribution. The computer system 601 may be configured to, for example, (i) identify optimal or beneficial microbial/mineral ratios, (ii) optimize one or more procedures for processing olivine to yield favorable properties or characteristics for the olivine, based on bioreactor conditions, and (iii) coordinate transportation of the olivine to the target site. The computer system 601 can be an electronic device of a user or a computer system that is remotely located with respect to the electronic device. The electronic device can be a mobile electronic device.

The computer system 601 may include a central processing unit (CPU, also “processor” and “computer processor” herein) 605, which can be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet. The network 630 in some cases is a telecommunication and/or data network. The network 630 can include one or more computer servers, which can enable distributed computing, such as cloud computing. The network 630, in some cases with the aid of the computer system 601, can implement a peer-to-peer network, which may enable devices coupled to the computer system 601 to behave as a client or a server.

The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some cases, the circuit is an application specific integrated circuit (ASIC).

The storage unit 615 can store files, such as drivers, libraries and saved programs. The storage unit 615 can store user data, e.g., user preferences and user programs. The computer system 601 in some cases can include one or more additional data storage units that are located external to the computer system 601 (e.g., on a remote server that is in communication with the computer system 601 through an intranet or the Internet).

The computer system 601 can communicate with one or more remote computer systems through the network 630. For instance, the computer system 601 can communicate with a remote computer system of a user (e.g., an end user performing or monitoring the processing and/or the transportation or distribution of the olivine). Examples of remote computer systems include personal computers (e.g., portable PC), slate or tablet PC's (e.g., Apple® iPad, Samsung® Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine readable code can be provided in the form of software. During use, the code can be executed by the processor 605. In some cases, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media including, for example, optical or magnetic disks, or any storage devices in any computer(s) or the like, may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, a portal for a user to monitor the processing and/or the transportation or distribution of the olivine. The portal may be provided through an application programming interface (API). A user or entity can also interact with various elements in the portal via the UI. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 605. For example, the algorithm may be configured to identify optimal or beneficial conditions and optimize a procedure for processing the olivine based on the properties or the characteristics of said conditions.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A system for processing an ultramafic material, comprising:
a reactor for accelerating weathering of said ultramafic material, wherein said reactor comprises one or more chambers comprising one or more microbes, one or more biological mediators, or one or more enzymatic accelerants to facilitate weathering of said ultramafic material.

2. The system of claim 1, wherein said one or more microbes comprises live or dead microbial cells or biomass.

3. The system of claim 2, wherein said live or dead microbial cells or biomass comprise heterotrophic, chemolitho-auotrophic or photo-autotrophic prokaryotes or eukaryotes.

4. The system of claim 1, wherein said one or more microbes comprise bacteria that are capable of growth in aqueous mediums.

5. The system of claim 4, wherein said bacteria comprises Alteromonas or Shewanella.

6. The system of claim 1, wherein said one or more microbes comprise eukaryotic algae capable of uptake of bacterially produced siderophores.

7. The system of claim 6, wherein said bacterially produced siderophores comprises Phaeodactylum or Thalassiosira diatom species.

8. The system of claim 1, wherein said one or more microbes comprise marine microbes capable of growing in saltwater.

9. The system of claim 1, wherein said one or more biological mediators comprises exo-metabolites such as ligands, redox-active compounds which may be related to quorum sensing or the electron transfer chain, or nucleic or amino acids.

10. The system of claim 1, wherein said one or more enzymatic accelerants comprises extra- or intracellular metalloenzymes, oxidoreductases, transferases, hydrolases, ligases or isomerases.

11. The system of claim 1, further comprising one or more mechanical components operably coupled to a chamber of said one or more chambers.

12. The system of claim 11, wherein said one or more mechanical components comprises pumps, or systems configured to provide fluid manipulation to transfer mechanical energy to the contents of the chamber.

13. The system of claim 12, wherein the systems configured to provide fluid manipulation are configured for fluid filtration, sterilization, cavitation, flow control, particulate recovery, flocculation, centrifugation, phase change reactions, pressurization or de-pressurization, fluid mixing, fluid separation, heat transfer, pressure release, ventilation, or management of sub-, trans-, super- and hyper-sonic fluid flows.

14. The system of claim 11, wherein said one or more mechanical components are operably coupled to a body of water within a zone comprising wave or current energy, such that said one or more mechanical components transfer mechanical energy from said body of water to said contents of said one or more chambers.

15. The system of claim 1, wherein said one or more chambers comprises a first chamber comprising said ultramafic material and a second chamber comprising said one or more microbes, one or more biological mediators, or one or more enzymatic accelerants to facilitate weather of said ultramafic material, and wherein said first chamber and said second chamber are operably coupled to mix at least a portion of said contents of said first chamber and said second chamber.

16. The system of claim 15, wherein said system further comprises a third chamber operably coupled to said first and second chamber, wherein said contents of said first and second chambers are at least partially mixed within said third chamber.

17. The system of claim 16, wherein said third chamber is in fluid communication with a body of water.

18. The system of claim 16, wherein said system further comprises a fourth chamber operably coupled to said third chamber, wherein technology metals are extracted from contents of said third chamber.

19. The system of claim 1, further comprising a controller and one or more sensors, wherein said one or more sensors are configured to collect data related to total carbon capture of said reactor, and wherein said controller is configured to adjust one or more operational parameters of said reactor for processing the ultramafic material based on said data of said one or more sensors.

20. The system of claim 1, wherein said system comprises a first chamber comprising said one or more microbes, one or more biological mediators, or one or more enzymatic accelerants.

21. The system of claims 1 to 20, wherein said ultramafic material comprises processed ultramafic materials.

22. The system of claim 21, wherein said ultramafic materials comprise mine wastes, pelletized olivine aggregates, or crushed olivine sand.
